(19) United States
(12) Patent Application Publication
(10) Pub. No.: US 2015/0044356 A1
(43) Pub. Date: Feb. 12, 2015

(54) Solid State Fermentation Systems and Process for Producing High-Quality Protein Concentrate and Lipids

(71) Applicant: Prairie AquaTech, Brookings, SD (US)

(72) Inventors: Jason A. Bootsma, Sioux Falls, SD (US); William R. Gibbons, Brookings, SD (US); Michael L. Brown, Volga, SD (US)

(73) Assignee: PRAIRIE AQUATECH, Brookings, SD (US)

(21) Appl. No.: 14/453,597

(22) Filed: Aug. 6, 2014

Related U.S. Application Data
(60) Provisional application No. 61/862,935, filed on Aug. 6, 2013.

(51) Int. Cl. A23K 1/00; A23K 1/14; C07K 2/00; A23K 1/18; C12P 7/64; A23K 1/06; A23J 1/12

(52) U.S. Cl. CPC A23K 1/007; A23K 1/06; A23K 1/14; A23J 1/125; A23K 1/188; C12P 7/6427; C07K 2/00
USPC 426/656; 426/531; 435/71.1; 435/71.2; 435/134; 554/224

(57) ABSTRACT

The present invention describes a bio-based process to produce high quality protein concentrate (HQPC) and lipids by converting plant derived materials into bioavailable protein and lipids via solid state fermentation (SSF) and hybrid-SSF, including the use of such HQPC and lipids so produced as nutrients, including use as a fish meal replacement in aquaculture diets. Also disclosed is a SSF reactor and method of using the reactor.

### CROSS REFERENCE TO RELATED APPLICATION

This application claims benefit under 35 U.S.C. §119(e) to U.S. Provisional Application No. 61/862,935, filed on Aug. 6, 2013, which is incorporated by reference herein in its entirety.

This work was made, in part, with Governmental support from the National Science Foundation under contract DBI-1005068. The Government may have certain rights in this invention.

### BACKGROUND OF THE INVENTION

1. Field of the Invention

The invention generally relates to fermentation processes, and specifically to solid state fermentation (SSF) processes to produce high quality protein concentrates and lipids, including SSF reactors, products made therefrom, and use of such products in the formulation of nutrient feeds.

2. Background Information

In 2008, approximately 28% of the world's wild, marine fish stocks were overexploited and 52% were fully exploited, even as the demand for per capita consumption of fish and shellfish products have increased with the increasing human population. With dwindling wild fish stocks, in an effort to meet this increased demand, commercial aquaculture production has increased dramatically. However, one of the primary constituents of dietary formulations for aquaculture, fish meal protein, is also derived from wild capture fisheries. It is estimated that at least 6.7 mmt of fish meal will be required to support commercial aquaculture production by 2012, and this is only expected to increase in the coming years. this is clearly an unsustainable trend.

Lower cost, more sustainable plant-derived sources of protein have been used to partially replace fish meal in aquaculture diets. Defatted soybean meal (SBM, 42-48% protein) has commonly been used to replace up to 29% of total protein in grower diets tor several species, while soy protein concentrate (SPC, 65% protein) has been tested successfully at higher total protein replacement levels, largely governed by the trophic states of the species. These soybean products provide high protein and relative good amino acid profiles, but are still deficient in some critical nutrients (e.g., taurine) required by carnivorous marine fishes. SPC can be used at higher levels than soybean meal, primarily because the solvent extraction process used to produce SPC removes anti-nutritional factors (e.g., oligosaccharides) and thereby increases protein bioavailability. In addition, a thermal step has been used to inactivate heat-labile antigenic factors. The primary limitations of the current solvent extraction process are its cost, the lack of use for the oligosaccharides removed in the process, and quality issues that frequently limit inclusion to 50% of total protein in the diet. Further, processing of soy material into soybean meal or soy protein concentrates can be environmentally problematic (e.g., problems with disposal of chemical waste associated with hexane-extraction), and final products may require supplementation with crude or refined fats where total fish meal replacement is contemplated.

Corn co-products, including dried distiller's grains with solubles (DDGS), have also been evaluated in aquaculture diets at fish meal replacement levels of up to 20%. DDGS has lower protein (28-32%) and more fiber than soy products, but is typically priced at ˜50% of the value of defatted soybean meal. Some ethanol plants have incorporated a dry fractionation process to remove part of the fiber and oil prior to the conversion process, resulting in a dry-frac DDGS of up to 42% protein. While this product has been used to replace 20-40% of fish meal in aquaculture feeds, there remains the need for a higher protein, more digestible DDGS aqua feed product. Such a product would be especially attractive if the protein component had higher levels of critical amino acids such as lysine, methionine, and cysteine.

In addition, microbial biomass derived lipid components are being contemplated as attractive renewable resources in the production of polyunsaturated fatty acids (PUFAs) and omega-3 fatty acids to supplement high protein feed and as a replacement for plant derived lipids lost during solvent stripping. Unfortunately, costs of producing microbial lipids containing polyenoic fatty acids, and especially the highly unsaturated fatty acids, such as C18:4n-3, C20:4n-6, C20:5n3, C22:5n-3, C22:5n-6 and C22:6n-3, have remained high in part due to the limited densities to which the high polyenoic fatty acid containing eukaryotic microbes have been grown and the limited oxygen availability both at the high cell concentrations and higher temperatures needed to achieve reasonable productivity.

Solid state fermentation (SSF) may be used to cultivate microorganisms for metabolic products and/or microbial altered substrates. SSF is defined as growth of microorganisms, usually fungi, on solid substrates in a defined gas phase, but in absence or near absence of free water phase. The past decade has witnessed an unprecedented interest in SSF for the development of bioprocesses such as bioremediation and biodegradation of hazardous compounds, biological detoxification of agro-industrial residues, biopulping and production of value-added products such as biologically active secondary metabolites, including antibiotics, alkaloids, plant growth factors, enzymes, organic acids, biosurfactants, aroma compounds, etc.

Traditional solid fermentation process technology has proved difficult and laborious to apply to modern biotechnical processes where strict asepsis may be required. In tray reactors the dead space is about one half of the bioreactor volume. The bioreactor size needed for particular product yield is therefore remarkably smaller in packed bed than in tray bioreactors, which make the tray type bioreactor less efficient. The operation of tray bioreactors also requires increased manual labor because each tray has to be filled, emptied, and cleaned individually.

By contrast, the packed bed bioreactor is easy to fill and empty by pouring the culture medium in and out and cleaning is also simple. The packed bed bioreactor is thus more cost, labor and space effective than the tray bioreactor. Drawbacks in packed bed reactors have been ensuring uniform inoculation and maintaining optimal incubation conditions.

Reactors with mixers have been developed for modern SSF applications but aseptic mixing devices equipped with motors can be very expensive. Mechanical abrasion in mixing may also damage the airy, loose structure of the growth medium when certain sensitive carriers are used. Rotating drum reactors can provide sufficient mixing only for solid growth media having a c certain kind of freely rolling structure.

Even novel solid state fermentations are still made using complex, bulky media such as cereal grains supplemented with various flours. Optimal control of growth conditions and product formation can be achieved on more defined media which can be sensitive to mixing or to immersing completely in liquid.

Increased interest in SSF exists because of certain advantages compared to submerged fermentation (SmF). Such advantages include effective production of secondary metabolites such as enzymes, aromatic substances as well as pharmaceutically active substances, or in the enrichment of lipids, proteins, vitamins or other nutritional products. However, in view of the above, there remains a need to generate high quality protein concentrates by processes that efficiently exploit the advantages offered by SSF.

### SUMMARY OF THE INVENTION

The present disclosure relates to an organic, microbially-based system to convert plant material into a highly digestible, concentrated protein source as well as polyunsaturated fatty acids (PUFA) via solid state fermentation (SSF), including such a concentrated source alone or in combination with said PUFA which source is suitable for use as a feed for animals used for human consumption, including a solid state fermentation reactor and methods of use. Further, a method which combines a submerged fermentation reaction with a SSF is also disclosed.

In embodiments, method of producing a non-animal based protein concentrate is disclosed including inoculating a substantially dry substrate including cereal grains, bran, sawdust, peat, oil-seed materials, wood chips, and combinations thereof; subjecting the inoculated substrate to solid state fermentation (SSF) with a microbe including Aureobasidium pullulans, Fusarium venenatum, Sclerotium glucanicum, Sphingomonas paucimobilis, Ralstonia eutropha, Rhodospirillum rubrum, Issatchenkia spp, Aspergillus spp, Kluyveromyces and Pichia spp, Trichoderma reesei, Pleurotus ostreatus, Rhizopus spp, and combinations thereof; incubating the inoculated substrate at a pH of less than about 2 to about 3 or at a pH of greater than about 8; and recovering the resulting proteins and microbes.

In one aspect, the method also includes mixing the microbe and substrate to form a substantially stable pellet or billet, wherein said pellet or billet contains sufficient void volume within and between pellets or billets to allow for aeration and humidification of the stabilized substrate-microbe mixture with substantially no agitation.

In a related aspect, the microbe is A. pullulans.

In another aspect, the substrate is non-extruded DDGS or non-extruded DDG.

In one embodiment, a protein concentrate produced by the method above is disclosed, where the protein content if the concentrate is between about 40 to about 50% (dry matter basis).

In another embodiment, the protein concentrate is included in a composition, which composition is a complete replacement for animal based fishmeal in a fish feed.

In one embodiment, a method of producing a non-animal based protein concentrate is disclosed including forming a feedstock and transferring the feedstock to a first bioreactor; inoculating the feedstock with at least one microbe in an aqueous medium, wherein said microbe converts released sugars into proteins and exopolysaccharides and optionally releases enzymes into the bulk fluid; mixing the liquid with an acid and optionally one or more antimicrobials; mixing additional solids to the mixture to reduce the moisture level of the mixture to about 40 to about 60% and transferring said reduced moisture mixture to a second bioreactor, where the mixture is then incubated in the second bioreactor for a sufficient time to convert the solids into said protein concentrate.

In one aspect, inoculating step is carried out at about 30 to about 50° C. for about 24 hours. In another aspect, missing of additional solids step is carried out at about 25° C. for about 5 days.

In a related aspect, the microbe is a fungus. In a further related aspect, the fungi is Aureobasidium pullulans.

In another aspect, the method includes supplementing the inoculum with a nitrogen source. In a related aspect, the nitrogen source includes ammonium sulfate, urea, and ammonium chloride.

In another aspect, the second bioreactor is conical or tubular.

In one aspect, the fermentation is carried out in the absence of exogenous saccharifying enzymes.

In one embodiment, a protein concentrate produced by the above method is disclosed, where the protein content is between about 50 to about 60% (dry matter basis).

In another embodiment, a composition including the protein concentrate above is disclosed, which composition is a complete replacement for animal based fishmeal in a fish feed.

In one embodiment, a method of producing a polyunsaturated fatty acid (PUFA) is disclosed including inoculating a substrate containing low PUFA lipids either as provided or by addition, where the substrate includes cereal grains, bran, sawdust, peat, oil-seed materials, wood chips, syrup, and combinations thereof; subjecting the inoculated substrate to solid state fermentation (SSF) with a microbe includes Pythium, Thraustochytrium and Schizochytritum, and combinations thereof; incubating the inoculated substrate.

In a related aspect, the method further includes adding the resulting PUFA enhanced material as an ingredient in an animal feed or alternatively recovering the resulting PUFA enhanced lipids.

In further related aspect, the produce of the above method is disclosed, where the lipid of the composition has about 50-90% triacylglycerol content.

### BRIEF DESCRIPTION OF THE DRAWINGS

FIG. 1 shows a schematic of the SSF reactor.

FIG. 2 shows Relative Growth, Feed Conversion Ratio, Fulton's Condition Factor (K), and Visceral Somatic Index (VSI) means at Day 112. Letters denote a significant difference between dietary treatments and error bars represent the standard error of the mean (SEM).

### DETAILED DESCRIPTION OF THE INVENTION

Before the present composition, methods, and methodology are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “lipid” includes one or more lipids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus ≦10% of particular term and “substantially” and “significantly” will mean plus or minus >10% of the particular term.

As used herein, “consisting essentially of” means, the particular component and may include other components, which other components do not change the novel properties or aspects of the particular component.

As used herein, the term “animal” means any organism belonging to the kindgom Animalia and includes, without limitation, humans, birds (e.g. poultry), mammals (e.g. cattle, swine, goat, sheep, cat, dog, mouse and horse) as well as aquaculture organisms such as fish (e.g. trout, salmon, perch), mollusks (e.g. clams) and crustaceans (e.g. lobster and shrimp).

Us of the term “fish” includes all vertebrate fishes, which may be bony (teleosts) or cartilaginous (chondrichthyes) fish species.

As used herein “non-animal based protein” means that the protein concentrate comprises at least 0.81 g of crude fiber/100 g of composition (dry matter basis), which crude fiber is chiefly cellulose, hemicellulose, and lignin material obtained as a residue in the chemical analysis of vegetable substances.

As used herein, “incubation process” means the provision of proper conditions for growth and development of bacteria or cells, where such bacteria or cells use biosynthetic pathways to metabolize various feed stocks. In embodiments, the incubation process may be carried out, for example, under aerobic conditions. In other embodiments, the incubation process may include anaerobic fermentation.

As used herein, the term “incubation products” means any residual substances directly resulting from an incubation process/reaction. In some instances, an incubation produce contains microorganisms such that it has a nutritional content enhanced as compared to an incubation product that is deficient in such microorganisms. The incubation products may contain suitable constituent(s) from an incubation broth. For example, the incubation products may include dissolved and/or suspended constituents from an incubation broth. The suspended constituents may include undissolved soluble constituents (e.g., where the solution is supersaturated with one or more components) and/or insoluble materials present in the incubation broth. The incubation products may include substantially all of the dry solids present at the end of an incubation (e.g., by spray drying an incubation broth and the biomass produced by the incubation) or may include a portion thereof. The incubation products may include crude material from incubation where a microorganisms may be fractionated and/or partially purified to increase the nutrient content of the material.

As used herein, a “conversion culture” means a culture of microorganisms which are contained in a medium that comprises material sufficient for the growth of the microorganisms, e.g., water and nutrients. The term “nutrient” means any substance with nutritional value. It can be part of an animal feed or food supplement for an animal. Exemplary nutrients include but are not limited to proteins, peptides, fats, fatty acids, lipids, water and fat soluble vitamins, essential amino acids, carbohydrates, sterols, enzymes, functional organic acids and trace minerals, such as, phosphorus, iron, copper, zinc, manganese, magnesium, cobalt, iodine, selenium, molybdenum, nickel, fluorine, vanadium, tin, and silicon.

Conversion is the process of culturing microorganisms in a conversion culture under conditions suitable to convert protein/carbohydrate/polysaccharide materials, for example, soybean material into a high-quality protein concentrate. Adequate conversion means utilization of 90% or more of specified carbohydrates to produce microbial cell mass and/or protein or lipid. In embodiments, conversion may be aerobic or anaerobic.

As used herein a “flocculent” or “clearing agent” is a chemical that promotes colloids to come out of suspension through aggregation, and includes, but is not limited to, a multivalent ion and polymer. In embodiments, such a flocculent/clearing agent may include bioflocculents such as exopolysaccharides.

As used herein, “hybrid-solid state fermentation”60 refers to a two step process comprising a first step where SMF or submerged fermentation (in an aqueous medium) is carried out in the presence of a microbe for about 24 hours to build up cell numbers as a source of inoculum, including where the inoculated microbe produces extracellular enzymes, with release of said enzymes into the bulk fluid, and where both cells and enzymes are available for reaction with the solids of the next step, which step comprises blending the above liquid with additional acid and antimicrobials (optionally), along with sufficient solids, to recued the moisture level of the mixture to about 40 to about 60%, where the latter becomes the solid phase state used for incubation in an SSF reactor. In embodiment, a 15% solids phase is run for 24 hours submerged, followed by the addition of solids to make a solid state substrate 50% solids, where the latter is run in that state for 5 days.

A large number of plant protein sources may be used in connection with the present disclosure as feed stocks for conversion. The main reason for using plant proteins in the feed industry is to replace more expensive protein sources, like animal protein sources. Another important factor is the danger of transmitting diseases thorough feeding animal proteins to animals of the same species. Examples for plant protein sources include, but are not limited to, protein from the plant family Fabaceae as exemplified by soybean and peanut, from the plant family Brassictaceae as exemplied by canola, cottonseed, the plant family Asteraceae including, but not limited to sunflower, and the plant family Arecaceae including copra. These protein sources, also commonly defined as oilseed proteins may be fed whole, but they are more commonly fed as a by-product after oils have been removed. Other plant protein sources include plant protein sources from the family Poaceae, also known as Gramineae, like cereals and grains especially corn, wheat and rice or other staple crops such as potato, cassava, and legumes (peas and beans), some milling by-products including germ meal or corn gluten meal, or distillery/brewery by-products. In embodiments, feed stocks for proteins include, but are not limited to, plant materials from soybeans, peanuts, Rapeseeds, barley, canola, sesame seeds, cottonseeds, palm kernels, grape seeds, olives, safflowers, sunflowers, copra, corn, coconuts, linseed, hazelnuts, wheat, rice, potatoes, cassavas, legumes, camelina seeds, mustard seeds, germ meal, corn gluten meal, distillery/brewery by-products, and combinations thereof.

In the fish farming industry the major fishmeal replacers with plant origin reportedly used, include, but are not limited to, soybean meal (SBM), maize gluten meal, Rapeseed/canola (Brassica sp.) meal, lupin (Lupinus sp. like the proteins in kernel meals of de-hulled white (Lupinus albus), sweet (L. angustifolius) and yellow (L. luteus) lupins, Sunflower (Helianthus annuus) seed meal, crystalline amino acids; as well as pea meal (Pisum sativum), Cottonseed (Gossypium sp.) meal, Peanut (groundnut; Arachis hypogaea) meal and oilcake, soybean protein concentrate, corn (Zea mays) gluten meal and wheat (Triticum aestivum) gluten, Potato (Solanum tuberosum L.) protein concentrate as well as other plant feedstuffs like Moringa (Moringa oleifera Lam.) leaves, all in various concentrations and combinations.

The protein sources may be in the form of non-treated plant materials and treated and/or extracted plant proteins. As an example, heat treated soy products have high protein digestibility.

A protein material includes any type of protein or peptide. In embodiments, soybean material or the like may be used such as whole soybeans. Whole soybeans may be standard, commoditized soybeans; soybeans that have been genetically modified (GM) in some manner; or non-GM identity preserved soybeans. Exemplary GM soybeans include, for example, soybeans engineered to produce carbohydrates other than stachyose and raffinose. Exemplary non-GM soybeans include, for example, Schillinger (Emerge) varieties that are line bred for low carbohydrates, low fat, and low trypsin inhibition.

Other types of soybean material include soy protein flour, soy protein concentrate, soybean meal and soy protein isolate, or mixtures thereof. The traditional processing of whole soybean into other forms of soy protein as soy protein flours, soy protein concentrates, soybean meal and soy protein isolates, includes cracking the cleaned, raw whole soybean into several pieces, typically six (6) to eight (8), to produce soy chips and hulls, which are then removed. Soy chips are then conditioned at about 60° C. and flaked to about 0.25 millimeter thickness. The resulting flakes are then extracted with an inert solvent, such as a hydrocarbon solvent, typically hexane, in one of several types of countercurrent extraction systems to remove the soybean oil. For soy protein flours, soy protein concentrates, and soy protein isolates, it is important that the flakes be desolventized in a manner which minimizes the amount of cooking or toasting of the soy protein to preserve a high content of water-soluble soy protein. This is typically accomplished by using vapour desolventizers or flash desolventizers. The flakes resulting from this process are generally referred to as “edible defatted flakes” or “white soy(bean) flakes.”

White soy beam flakes, which are the starting material for soy protein flour, soy protein concentrate, and soy protein isolate, have a protein content of approximately 50%. White soybean flakes are then milled, usually in an open-loop grinding system, by a hammer mill, classifier mill, roller mill or impact pin mill first into grits, and with additional grinding, into soy flours with desired particle sizes. Screening is typically used to size the product to uniform particle size ranges, and can be accomplished with shaker screens or cylindrical centrifugal screeners. Other oil seeds may be processed in a similar manner.

In embodiments, distiller's dried grain solubles (DDGS) may be used DDGS are currently manufactured by the corn ethanol industry. Traditional DDGS comes from dry grind facilities, in which the entire corn kernel is ground and processed. DDGS in these facilities (e.g., “front end” fermentation) typically contains 28-32% protein and between about 9 to about 13% crude fat. However, in “back end” oil extraction, about ⅓ of the corn oil is extracted from, e.g., thin stillage, prior to producing “reduced-oil” DDGS (containing about 5 to about 9% crude fat), which has slightly more protein and fiber relative to DDGS produced without oil extraction. In a related aspect, either reduced oil or traditional DDGS may be used.

The protein sources may be in the form of non-treated plant materials and treated and/or extracted plant proteins. As an example, heat treated soy products have high protein digestibility. Still, the upper inclusion level for full fat or defatted soy meal inclusion in diets for carnivorous fish is between an inclusion level of 20 to 30%, even if heat labile antinutrients are eliminated. In fish, soybean protein has shown that feeding fish with protein concentration inclusion levels over 30% causes intestinal damage and in general reduces growth performance in different fish species. In fact, most farmers are reluctant to use more than 10% plant proteins in the total diet due to these effects.

The present invention solves this problem and allows for plant protein inclusion levels of up to 40 or even 50%, depending on, amongst other factors, the animal species being fed, the origin of the plant protein source, the ratio of different plant protein sources, the protein concentration and the amount, origin, molecular structure and concentration of the glucan and/or mamman. In embodiments, the plant protein inclusion levels are up to 40%, preferably up to 20 or 30%. Typically the plant protein present in the diet is between 5 and 40%, preferably between 10 or 15 and 30%. These percentages define the percentage amount of a total plant protein source in the animal feed or diet, this includes fats, ashes etc. In embodiments, pure protein levels are up to 50%, typically up to 45%, in embodiments 5-95%.

The proportion of plant protein to other protein in the total feed or diet may be 5:95 to 95:5, 15:85 to 50:50, or 25:75 to 45:55.

The disclosed microorganisms must be capable of converting carbohydrates and other nutrients into a high-quality protein concentrate in a conversion culture. In embodiments, the microorganism is a yeast-like fungus. An example of a yeast-like fungus is Aurobasidium pullulans. Other example microorganisms include yeast such as kluyveromyces and Pichia spp, Lactic acid bacteria, Trichoderma reesei, Pleurotus ostreatus, Rhizopus spp, and many types of lignocellulose degrading microbes. Generally, exemplary microbes include those microbes that can metabolize stachyose, raffinose, xylose and other sugars. However, it is within the abilities of a skilled artisan to pick, without undue experimentation, other appropriate microorganisms based on the disclosed methods.

In embodiments, the microbial organisms that may be used in the present process include, but are not limited to, Aureobasidium pullulans, Fusarium venenatum, Sclerotium glucanicum, Sphingomonas paucimobilis, Ralstonia eutropha, Rhodospirillum rubrum, Issatchenkia spp, Penicillium spp, Kluyveromyces and Pichia spp, Trichoderma reesei, Pleurotus ostreatus, Rhizopus spp, and combinations thereof. In embodiments, the microbe is Aureobasidium pullulans.

In embodiments, the A. pullulans is adapted to various environments/stressors encountered during conversion. In embodiments, an A. pullulans strain denoted by NRRL deposit No. 50793, which was deposited with the Agricultural Research Culture Collection (NRRL), Peoria, Ill., under the terms of the Budapest Treaty on Nov. 30, 2012, exhibits lower gum production and is adapted to DDGS and SBM based media. In embodiments, an A. pullulans strain denoted by NRRL deposit No. 50792, which was deposited with the Agricultural Research Culture Collection (NRRL), Peoria, Ill., under the terms of the Budapest Treaty on Nov. 30, 2012, is adapted to high levels of the antibiotic tetracycline (e.g., from about 75 μg/ml tetracycline to about 200 μl/ml tetracycline). In embodiments, an A. pullulans strain denoted by NRRL deposit No. 50794, which was deposited with the Agricultural Research Culture Collection (NRRL), Peoria, Ill., under the terms of the Budapest Treaty on Nov. 30, 2012, is adapted to high levels of the antibiotic LACTROL® (e.g., from about 2 μg/ml virginiamycin to about 6 μg/ml virginiamycin). In embodiments, an A. pullulans strain denoted by NRRL deposit No. 50795, which was deposited with the Agricultural Research Culture Collection (NRRL), Peoria, Ill., under the terms of the Budapest Treaty on Nov. 30, 2012, is acclimated to condensed corn solubles.

In other embodiments, an A. pullulans strain may be acclimated to 450-550 ppm LACTROL® (e.g., virginiamycin). In embodiments, an A. pullulans strain may be acclimated to pH 1.5-1.75. In embodiments, an A. pullulans strain may be acclimated to 90-110 ppm Isostab. In embodiments, an A. pullulans strain may be acclimated to 80-100 ppm Betastab. In embodiments, an A. pullulans strain may produce cellulase enzymes and may be acclimated to soybean meal and DDGS. In embodiments, the A. pullulans is selected from NRRL 42023, NRRL 58522 or Y-2311-1.

In other embodiments, a Thermotolerant Pichia strain may be acclimated to soybean meal and DDGS.

In embodiments, an Issatchenkia spp strain may be acclimated to soybean meal and DDGS.

In embodiments, a Fusarium venenatum strain may produce cellulase enzymes and may be acclimated to soybean meal and DDGS.

In other embodiments, a Pennicillium spp strain may produce cellulase enzymes and may be acclimated to soybean meal and DDGS.

In embodiments, Aspergillus orzyae strain may be acclimated to soybean meal and DDGS.

In embodiments, microorganisms which are capable of producing lipids comprising omega-3 and/or omega-6 polyunsaturated fatty acids include those microorganisms which are capable of producing DHA. In a related aspect, such organisms include marine microorganisms, for example algae, such as Thraustochytrids of the order Thraustochytriales, more specifically Traustochytriales of the genus Traustochytrium and Schizochytrium, including Traustochytriales which are disclosed in U.S. Pat. Nos. 5,340,594 and 5,340,742, the disclosures of which are incorporated herein by reference in their entireties. It is to be understood, however, that the invention as a whole is not intended to be so limited, and that one skilled in the art will recognize that the concept of the present invention will be applicable to other microorganisms producing a variety of other compounds, including other lipid compositions, in accordance with the techniques discussed herein.

As used herein a “fatty acid” means an aliphatic monocarboxylic acid. Lipids are recognized to be fats or oils including the glyceride esters of fatty acids along with associated phosphatides, sterols, alcohols, hydrocarbons, ketones, and related compounds.

A commonly employed shorthand system is used in this disclosure to denote the structure of the fatty acids (e.g., Weete, “Lipid Biochemistry of Fungi and Other Organisms”, Plenum Press, New York (1980)). This system uses the letter “C” accompanied by a number denoting the number of carbons in the hydrocarbon chain, followed by a colon and a number indicating the number of double bonds, e.g., C20:5, eicosapentaenoic acid. Fatty acids are numbered starting at the carboxy carbon. Position of the double bonds is indicated by adding the Greek letter delta (Δ) followed by the carbon number of the double bond; i.e., C20:5omega-3 Δ^{5,8,11,14,17}. The “omega” notation is a shorthand system for unsaturated fatty acids whereby numbering from the carboxy-terminal carbon is used. For convenience, ω3 will be used to symbolize “omega-3”, especially when using the numerical shorthand nomenclature described herein. Omega-3 highly unsaturated fatty acids are understood to be polyethylenic fatty acids in which the ultimate ethylenic bond is 3 carbons from and including the terminal methyl group of the fatty acid. Thus, the complete nomenclature for eicosapentaenoic acid, an omega-3 highly unsaturated fatty acid, would be C20:5ω3Δ^{5,8,11,14,17}. For the sake of brevity, the double bond locations (Δ^{5,8,11,14,17}) will be omitted. Eicosapentaenoic acid is then designated C20:5ω3, Docosapentaenoic acid (C22:5ω3Δ^{7,10,13,16,19}) is C22:5ω3, and Docosahexaenoic acid (C22:6ω3Δ^{4,7,10,13,16,19}) is C22:6ω3. The nomenclature “highly unsaturated fatty acid” means a fatty acid with 4 or more double bonds. “Saturated fatty acid” means a fatty acid with 1 to 3 double bonds.

Desirable characteristics of the organisms for the production of omega-3 highly unsaturated fatty acids include, but are not limited to those: 1) capable of heterotrophic growth; 2) high content of omega-3 highly unsaturated fatty acids; 3) unicellular; 4) low content of saturated and omega-6 highly unsaturated fatty acids; 5) thermotolerant (ability to grow at temperatures above 30° C.); and 6) euryhaline (able to grow over a wide range of salinities, including low salinities).

Lipids may comprise one or more of the following compounds: lipstatin, statin, TAPS, pimaricine, nystatine, fat-soluble antibiotic (e.g., laidlomycin) fat-soluble anti-oxidant (e.g., co-enzyme Q10), cholesterol, phytosterol, desmosterol, tocotrienol, tocopherol, carotenoid, or xanthophylls, for instance beta-carotene, lutein, lycopene, astaxanthin, zeaxanthin, or canthaxanthin, fatty acids, such as conjugated linoleic acids or polyunsaturated fatty acids (PUFAs). In embodiments, the lipid comprises at least one of the compounds mentioned above at a concentration of at about 5 wt. % or at least about 10 wt. % (with respect to the weight of the lipid).

Lipids may be obtained comprising for example triglyceride, phospholipid, free fatty acid, fatty acid ester (e.g., methyl or ethyl ester) and/or combinations thereof. In embodiments, lipids have a tricylglycerol content of at least about 50%, at least about 70%, or at least about 90%.

In embodiments, a lipid comprises a polyunsaturated fatty acid (PUFA), for instance a PUFA having at least 18 carbon atoms, for instance a C₁₈, C₂₀ or C₂₂ PUFA. In embodiments, the PUFA is an omega-3 PUFA (ω3) or an omega-6 PUFA (ω6). In related aspects, the PUFA has at least 3 double bonds. In embodiments, PUFAs are: docosahexaenoic acid (DHA, 22:6 ω3); γ-linolenic acid (GLA, 18:3 ω6); α-linolenic acid (ALA, 18:3 ω3); dihomo-γ-linolenic acid (DGLA, 20:3 ω6); arachidonic acid (ARA, 20:4 ω6); and eicosapentaenoic acid (EPA, 20:5 ω3).

In embodiments, a lipid comprises at least one PUFA (for instance ARA or DHA) at a concentration of at least about 5 wt. %, for instance at least about 10 wt. %, for instance at least about 20 wt. % (with respect to the weight of the lipid).

The PUFA may be in the form of a (mono-, di, or tri) glyceride, phospholipid, free fatty acid, fatty acid ester (e.g. methyl or ethyl ester) and/or combinations thereof. In a related aspect, a lipid is obtained wherein at least about 50% of all PUFAs are in triglyceride form.

The lipid may be an oil or fat, for instance an oil comprising a PUFA.

The cells may be any cells comprising a lipid. Typically, the cells have produced the lipid. The cells may be whole cells or ruptured cells. The cells may be of any suitable origin. The cells may for instance be plant cells, for instance cells from seeds or cells of a microorganism (microbial cells or microbes). Examples of microbial cells or microbes are yeast cell, bacterial cells, fungal cells, and algal cells. In embodiments, fungi may be use, for example, such as the order Mucorales, for example, Mortierella, Phycomyces, Blakeslea, Aspergillus, Thraustochytrium, Phythium or Entomophthora. In embodiments, a source of arachidonic acid (ARA) may be from Mortierella alpina, Blakeslea trispora, Aspergillus terreus or Pythium insidiosum. Algae may be dinoflagellate and/or include Porphyridium, Nitszchia, or Crypthecodinium (e.g. Crypthecodinium cohnii). Yeasts may include those of the genus Pichia or Saccharomyces, such as Pichia ciferii. Bacteria may be of the genus Propionibacterium. Examples of plant cells comprising a lipid are cells from soy bean, rape seed, canola, sunflower, coconut, flax and palm seed. In embodiments, the cells are plant cells comprising lipid which lipid comprises ARA. In embodimens, the cells as disclosed may be used alone or in combination.

In embodiments, the cells are used in fermentation.

In embodiments, the process according to the disclosure comprises one or more of the following steps: (i) heating or pasteurizing the cells; (ii) separating water from the cells by mechanical separation; (iii) washing the cells; and (iv) squeezing the cells.

Heating or pasteurizing may be effected at a temperature of from about 65° C. to about 120° C. It may inactivate or denature enzymes such as lipases and/or lipoxygenases.

Separating water from the cells by mechanical separation may be used to obtain the values for the water content and/or dry matter content as disclosed herein. Mechanical separation may for instance involve filtering, certrifuging, squeezing, sedimentation, or the use of a hydrocyclone.

The lipid may further be treated in any suitable manner. If the lipid is recovered by extraction with a solvent, the lipid may be obtained from the solvent by evaporation of the solvent.

The lipid obtained or obtainable by the processes according to the present disclosure may be subjected to further treatments, for instance to acid treatment (also referred to as degumming), alkali treatment (also referred to as neutralization), bleaching, deodorizing, cooling (also referred to as winterization).

The lipid obtained or obtainable by the process according to the present disclosure has many uses. It may for instance be used for the preparation of a food product, for instance a human food product (e.g., infant formula), or an animal feed product. It may also be used for the preparation of a pharmaceutical product or a cosmetic product. Accordingly, the disclosure also provides a food product (e.g., fortified food or a nutritional supplement), for instance a human food product (e.g., infant formula), or an animal feed product, a pharmaceutical product, a cosmetic product, comprising the lipid obtained or obtainable by the process according to the disclosure.

### Conversion Culture

In exemplary embodiments, after pretreatment, the protein material (such as extruded soy while flakes) may be blended with water at a solid loading rate of at least about 5%, with pH adjusted to about 4.5-5.5. Then appropriate dosages of hydrolytic enzymes may be added and the slurry incubated with agitation at about 50-250 rpm at about 50° C. for about 3-24 h. After cooling to about 35° C., an inoculum of A. pullulans may be added and the culture may be incubated for an additional 72-120 h, or until the carbohydrates are consumed. during incubation, sterile air may be sparged into the reactor at a rate of about 0.5-1 L/L/h. In embodiments, the conversion culture undergoes conversion by incubation with the soybean material for less than about 96 hours. In embodimens, the conversion culture will be incubated for between about 96 hours and about 120 hours. In embodiments, the conversion culture may be incubated for more than about 120 hours. The conversion culture may be incubated at about 35° C.

In embodiments, the pH of the conversion culture, while undergoing conversion, may be about 4.5 to about 5.5. In embodiments, the pH of the conversion culture may be less than 4.5 (e.g., at pH 3). In embodiments, the conversion culture may be actively aerated such as is disclosed in Deshpande et al., Aureobasidiumpullulans in applied microbiology: A status report, Enzyme and Microbial Technology (1992), 14(7):514.

The high-quality protein concentrate (HQPC), as well as pullulan and siderophores, may be recovered from the conversion culture following the conversion process by optionally alcohol precipitation and centrifugation. An example alcohol is ethanol, although the skilled artisan understands that other alcohols should work. In embodiments, salts may also be used to precipitate. Exemplary salts may be salts of potassium, sodium and magnesium chloride. In embodiments, a polymer or multivalent ions may be used alone or in combination with the alcohol.

In embodiments, final protein concentrations solids recovery may be modulated by varying incubation times. For example, about 75% protein may be achieved with a 14 day incubation, where the solids recovery is about 16-20%. In embodiments, incubation for 2-2.5 days increase solids recovery to about 60-64%, and protein level of 58-60% in the HQPC. In embodiments, 4-5 day incubation may maximize both protein content (e.g., but not limited to greater than about 70%) and solids recovery (e.g., but not limited to greater than about 60%). These numbers may greater or lower, depending on the feed stock. In embodiments, the protein concentrates (i.e., HQSPC or HP-DDGS) may have a specific lipid:protein ratio, e.g., at about 0.010:1 to about 0.03:1, about 0.020:1 to about 0.025:1 or about 0.021:1 to about 0.023:1.

In embodiments, feed stocks may be extruded in a single screw extruder (e.g., BRABENDER PLASTI-CORDER EXTRUDER Model PL2000, Hackensack, N.J.) with a barrel length to screw diameter of 1:20 and a compression ratio of 3:1, although other geometries and ratios may be used. Feed stocks may be adjusted to about 10% to about 15% moisture, to about 15%, or to about 25% moisture. The temperature of feed, barrel, and outlet sections of extruder may be held at between about 40° C. to about 50° C. or to about 50° C. to about 100° C., about 100° C. to about 150° C., about 150° C. to about 170° C., and screw speed may be set at about 50 rpm to about 75 rpm or about 75 rpm to about 100 rpm or about 100 rpm to about 200 rpm to about 250 rpm. In embodiments, the screw speed is sufficient to provide a shearing effect against the ridged channels on both sides of a barrel. In embodiments, screw speed is selected to maximize sugar release.

In embodiments, extruded feed stock materials (e.g., plant proteins or DDGS) may be mixed with water to achieve a solid loading rate of at least 5% in a reactor (e.g., a 5 L NEW BRUNSWICK BIOFLO 3 BIOREACTOR; 3-4 L working volume). The slurry may be autoclaved, cooled, and then saccharified by subjection to enzymatic hydrolysis using a cocktail of enzymes including, but not limited to, endo-xylanase and beta-xylosidase, Glycoside Hydrolase, β-glucosidases, hemicellulase activities. In one aspect, the cocktail of enzymes includes NOVOZYME® enzymes. Dosages to be may include 6% CELLICCTEK® (per gm glucan), 0.3% CELLICHTEK® (per gm total solids), and 0.15% NOVOZYME 960® (per gm total solids). Saccharification may be conducted for about 12 h to about 24 h at 40° to about 50° C. and about 150 rpm to about 200 rpm to solubilize the fibers and oligosaccharides into simple sugars. The temperature may then be reduced to between about 30° C. to about 37° C., in embodiments to about 35° C., and the slurry may be inoculated with 2% (v/v) of a 24 h culture of the microbe. The slurry may be aerated at 0.5 L/L/min and incubation may be continued until sugar utilization ceases or about 96 h to about 120 h. In fed-batch conversions more extruded feed stock may be added during either saccharification and/or the microbial conversion phase.

In embodiments, the feed stock and/or extrudate may be treated with one or more antibiotics (e.g., but not limited to, tetracycline, penicillin, erythromycin, tylosin, virginiamycin, and combinations thereof) before inoculation with the converting microbe to avoid, for example, contamination by unwanted bacteria strains.

During incubation, samples may be removed at 6-12 h intervals. Samples for HPLC analysis may be boiled, centrifuged, filtered (e.g., through 0.22-μm filters), placed into autosampler vials, and frozen until analysis. In embodiments, samples may be assayed for carbohydrates and organic solvents using a WATERS HPLC system, although other HPLC systems may be used. Samples may be subjected to plate or helocytometer counts to assess microbial populations. Samples may also be assayed for levels of cellulose, hemicellulose, and pectin using National Renewable Energy Laboratory procedures.

In embodiments, the conversion culture may be combined with a lipid generating microorganism and/or the product of the lipid generating culture may be combined with the product of the conversion culture. In a related aspect, the lipid generating microorganism may be grown in a separate SmF process. In a further related aspect, the lipid generating microorganism may be Thraustochytrium aureum, where the substrate is syrup, and where the organisms tolerates salt water, including tolerating the high salt and high fat content of syrup.

### SSF

According to the method of the present disclosure, the solid growth medium inside the solid state fermenting (SSF) reactor may be used for the production of, inter alia, food stuffs for animal feed.

When a controlled mass flow of the solid growth media passes the point of inoculation it may be uniformly and continuously inoculated. The solid growth medium may comprise various organic or inorganic carriers, which may be moved by traveling vertical agitation, where auger sections may lift the fermentation substrate to increase aeration, distribute heat, distribute moisture, prevent clumping and packing of the substrate.

The inorganic carriers may include, but are not limited to, vermiculite, perlite, amorphous silica or granular clay. These types of materials are commonly used because they form loose, airy granular structure having a particle size of 0.5-50 mm and a high surface area. The organic carriers may include, but are not limited to, cereal grains, bran, sawdust, peat, oil-seed materials, wood chips, or combinations thereof. In a related aspect, these carriers may be separated from the final protein product.

In addition, the solid growth medium may contain supplemental nutrients for the microorganism. Typically, these include carbon sources such as carbohydrates (sugars, starch), proteins or fats, nitrogen sources in organic form (proteins, amino acids) or inorganic nitrogen salts (ammonium and nitrate salts, urea), trace elements or other growth factors (vitamins, pH regulators). The solid growth medium may contain aids for structural composition, such as super absorbents, for example polyacrylamides. It will be apparent to one of skill in the art that nutrient concentration, moisture content, pH, and the like may be modulated to optimize growth.

In embodiments, the solid growth medium may be sterile. For example, traveling vertical agitation bed may be detached from the reactor body, filled with solid growth medium and sterilized in, e.g., an autoclave, after which it may again be attached to the reactor body aseptically before starting the operation. In other embodiments, bacterial growth may be prevented, and autoclaving replaced, by the addition of a stabilized chorine dioxide product (e.g., FERMASURE™, from E.I. DuPont De Nemours and Co., Wilmington, Del.) or other antibacterial alternatives approved for safe human and animal consumption, including but not limited to, hydrogen peroxide, phosphorus, hydrochloric acid, tetracycyline, and synthetic antimicrobials (see, e.g., U.S. Pub. No. 20130084615, herein incorporated by reference in its entirety).

In embodiments, the solid growth medium inside the medium sterilizing unit is sterilized in situ before starting the inoculation, e.g., with the aid of steam. In other embodiments, the medium may be pasteurized or optionally no heat at all added, where the use of low water activity and low pH may be exploited to control bacterial growth.

The inoculum may be fed to the reactor according to the invention in liquid or solid form.

If liquid media is used as inoculum, it may be in the form of, for example, a suspension with a small particle size to enable the use of spraying techniques. In embodiments, the liquid media may be sprayed on a continuous stream of the solid growth medium passing the point of inoculation.

If the inoculum is in solid form, it may be transported to the point of inoculation similarly to transporting the solid growth medium, by vertical agitation/auger. In embodiments, the solid inoculum may be transported using a screw or belt conveyor. This ensures that the microorganism may be transported equally for cultivation. In other embodiments, the substrate and inoculum may be mixed and passed through a low temperature extruder to create stable pellets, where such pellets would allow for more effective air flow in the reactor in the absence of mechanical agitation.

There may be several different constructions to realize the functionality of the disclosed SSF.

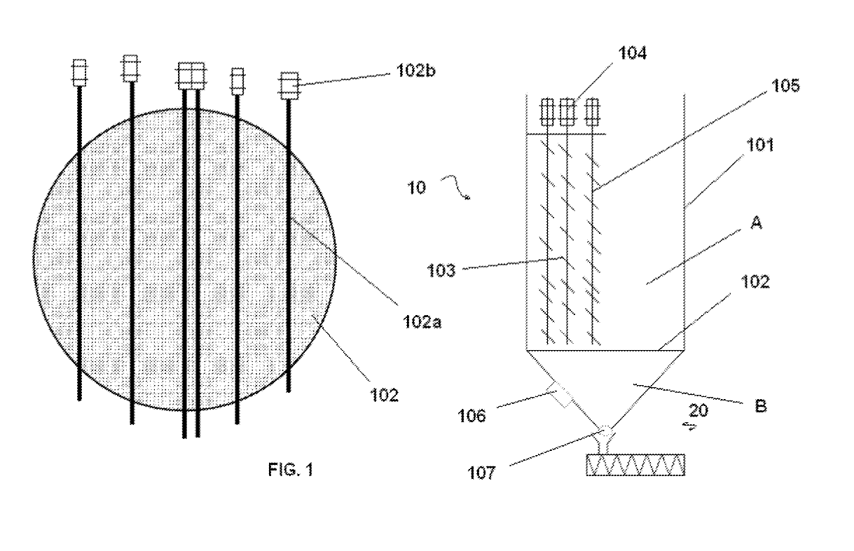

In embodiments, an SSF system is disclosed including reactor body 101 of reactor 10 comprising the entities as shown in FIG. 1. There are two main compartments, an upper compartment “A” and a cone-shaped bottom “B”, separated from each other by perforated “false” floor 102, which perforated floor 102 is configured in segments that rotate in a downward direction such that the floor 102 substantially opens up to allow material to flow down and be discharged out of reactor 10 to cone-shaped bottom B via gravity, and a plurality of traveling mixing screws 103 having positioners 104 attached to shafts 105, which screws 103 are configured to move horizontally throughout reactor body 101. Floor 102 movement is controlled by a plurality of axial rods 102a with positioner 102b. The cone-shaped bottom B comprises aeration input 106 and product output 107, which discharged material may be loaded onto a conveyor or separate auger-type device 20 for movement away from reactor 10. In embodiments, material on the mixing screws contains the discharged material to be deposited after sufficient fermentation.

In embodiments, reactor 10 is configured to accept temperature controlled humidified air in to cone-shaped bottom B under floor 102. Such a configuration provides the necessary oxygen to the microbe, removes heat, and controls moisture.

In one aspect, hot air is introduced at the end of the fermentation cycle. This allows, in combination with of the aeration floor 102 and vertical agitation via mixing screws 103, a method to dry the product down to the final desired moisture.

the nature of the fermentation process, as disclosed herein, is that it allows for drying in fermentation reactor 10. The use of fermentation reactor 10 also allows for more efficient drying of the protein product at low temperature, which also affords maintenance of enzymatic activity in the product. Further, the use of aeration drying is more efficient and saves energy because it takes advantage of physical and thermodynamic properties of gas-vapor mixtures (i.e., psychrometrics). Drying of the product in reactor 10 also provides for improved flow-ability and will allow the product to discharge by gravity, since it avoids handling and conveying of high moisture content materials. Moreover, device 10 as disclosed avoids the use of a separate drying system and associated conveyors, controls, and accompanying large foot prints.

In embodiments, mixing and abatement of stratification in the bed are provided such that reduction in clumping and agglomeration are achieved. In other embodiments, the rate and pattern of the horizontal travel throughout the upper compartment is programmable and selectable for any desired condition. In embodiments, the reactor air input contains a selectable temperature and humidity level. In one aspect, after the selected incubation period, the humidity may be reduced and the temperature increased to provide drying of the material and assist in the discharging process. The shape and size of the reactor compartments may vary depending on the need of the cultivation and used materials. The shape needs not to be restricted to well defined shapes, but may be moldable or plastic like. In embodiments, the shapes of the vessels are cylindrical, angular or conical.

In embodiments, SSF and SmF may be used serially, in any order, to produce the final product.

In embodiment, SSF and SmF are combined to achieve hybrid solid state fermentation (hybrid-SSF). SMF or submerged fermentation is carried out for about 24 hours to build up cell numbers as a source of inoculum, including where the inoculated microbe produces extracellular enzymes, with release of said enzymes into the bulk fluid, and where both cells and enzymes are available for reaction with the solids of the next step, which step comprises blending the above liquid with additional acid and antimicrobials (as needed), along with sufficient solids, to reduce the moisture level of the mixture to about 40 to about 60%, where the latter becomes the solid phase state used for incubation in the SSF reactor. In embodiment, a 15% solids is run for 24 hours submerged, followed by the addition of solids to make a solid state substrate 50% solids, where the latter is run in that state for 5 days.

### Dietary Formulations

In exemplary embodiments, the high-quality protein concentrate and lipids recovered are used in dietary formulations. In embodiments, the recovered high-quality protein concentrate (HQPC) will be the only protein source in the dietary formulation. Protein source percentages in dietary formulations are not meant to be limiting and may include 24 to 80% protein. In embodiments, the high-quality protein concentrate (HQPC) will be more than about 50%, more than about 60%, or more than about 70% of the total dietary formulation protein source. Recovered HQPC/lipid combinations may replace sources such as fish meal, soybean meal, wheat and corn flours and glutens and concentrates, and animal byproduct such as blood, poultry, and feather meals. Dietary formulations using recovered HQPC/lipids may also include supplements such as mineral and vitamin premixes to satisfy remaining nutrient requirements as appropriate.

In certain embodiments, performance of the HQPC, such as high-quality soy protein concentrate (HQSPC) or high-quality DDGS (HP-DDGS) or other upgraded plant-based meals alone or in combination with generated lipids, may be measured by comparing the growth, feed conversion, protein efficiency, and survival of animal on a high-quality protein concentrate dietary formulation to animals fed control dietary formulations, such as fish-meal. In embodiments, test formulations contain consistent protein, lipid, and energy contents. For example, when the animal is a fish, viscera (fat deposition) and organ (liver and spleen) characteristics, dress-out percentage, and fillet proximate analysis, as well as intestinal histology (enteritis) may be measured to assess dietary response.

As is understood, individual dietary formulations containing the recovered HQPC and/or combinations with recovered lipids may be optimized for different kinds of animals. In embodiments, the animals are fish grown in commercial aquaculture. Methods for optimization of dietary formulations are well-known and easily ascertainable by the skilled artisan without undue experimentation.

Complete grower diets may be formulated using HQPC in accordance with known nutrient requirements for various animal species. In embodiments, the formulation may be used for yellow perch (e.g., 42% protein, 8% lipid). In embodiments, the formulation may be used for rainbow trout (35% protein, 16% lipid). In embodiments, the formulation may be used for any one of the animals recited supra.

Basal mineral and vitamin premixes for plant-based diets may be used to ensure that micro-nutrient requirements will be met. Any supplements (as deemed necessary by analysis) may be evaluated by comparing to an identical formulation without supplementation; thus, the feeding trial may be done in a factorial design to account for supplementation effects. In embodiments, feeding trials may include a fish meal-based control diet and ESPC- and LSPC-based reference diets [traditional SPC (TSPC) is produced from solvent washing soy flake to remove soluble carbohydrate; texturized SPC (ESPC) is produced by extruding TSPC under moist, high temperature; and low-antigen SPC (LSPC) is produced from TSPC by altering the solvent wash and temperature during processing]. Pellets for feeding trials may be produced using the lab-scale single screw extruder (e.g., BRABENDERPLASTI-CORDER EXTRUDER Model PL2000).

### Feeding Trials

In embodiments, a replication of four experimental units per treatment (i.e., each experimental and control diet blend) may be used (e.g., about 60 to 120 days each). Trials may be carried out in 110-L circular tanks (20 fish/tank) connected in parallel to a closed-loop recirculation system driven by a centrifugal pump and consisting of a solids sump, and bioreactor, filters (100 μm bag, carbon and ultra-violet). Heat pumps may be used as required to maintain optimal temperatures for species-specific growth. Water quality (e.g., dissolved oxygen, pH, temperature, ammonia and nitrite) may be monitored in all systems.

In embodiments, experimental diets may be delivered according to fish size and split into two to five daily feedings. Growth performance may be determined by total mass measurements taken at one to four weeks (depending upon fish size and trial duration); rations may be adjusted in accordance with gains to allow satiation feeding and to reduce waste streams. Consumption may be assessed biweekly from collections of uneaten feed from individual tanks. Uneaten feed may be dried to a constant temperature, cooled, and weighed to estimate feed conversion efficiency. Feed protein and energy digestibilities may be determined from fecal material manually stripped during the midpoint of each experiment or via necropsy from the lower intestinal tract at the conclusion of a feeding trial. Survival, weight gain, growth rate, health indices, feed conversion, protein and energy digestibilities, and protein efficiency may be compared among treatment groups. Proximate analysis of necropsied fishes may be carried out to compare composition of fillets among dietary treatments. Analysis of amino and fatty acids may be done as needed for fillet constituents, according to the feeding trial objective. Feeding trial responses of dietary treatments may be compared to a control (e.g., fish meal) diet response to ascertain whether performance of HQPC diets meet or exceed control responses.

Statistical analyses of diets and feeding trial responses may be completed with an a priori α=0.05. Analysis of performance parameters among treatments may be performed with appropriate analysis of variance or covariance (Proc Mixed) and post hoc multiple comparisons, as needed. Analysis of fish performance and tissue responses may be assessed by nonlinear models.

In embodiments, the present disclosure proposes to convert fibers and other carbohydrates in soy flakes/meal or DDGS into additional protein using, for example, a GRAS-status microbe. A microbial exopolysaccharide (i.e., gum) may also be produced that may facilitate extruded feed pellet formation, negating the need for binders. This microbial gum may also provide immunostimulant activity to activate innate defense mechanisms that protect fish from common pathogens resulting from stressors. Immunoprophylactic substances, such as β-glucans, bacterial products, and plant constituents, are increasingly used in commercial feeds to reduce economic losses due to infectious diseases and minimize antibiotic use. The microbes of the present disclosure also produce extracellular peptidases, which should increase corn protein digestibility and absorption during metabolism, providing higher feed efficiency and yields. As disclosed herein, this microbial incubation process provides a valuable, sustainable aquaculture feed that is less expensive per unit of protein than SBM, SPC, and fish meal.

As disclosed, the instant microbes may metabolize the individual carbohydrates in soy flakes/meal or DDGS, producing both cell mass (protein) and a microbial gum. Various strains of these microbes also enhance fiber deconstruction. The microbes of the present invention may also convert soy and corn proteins into more digestible peptides and amino acids. In embodiments, the following actions in may be performed: 1) determining the efficiency of using select microbes of the present disclosure to convert pretreated soy protein, oil seed proteins, DDGS and the like, yielding a high quality protein concentrate (HQPC) with a protein concentration of between about 45% and 55% or at least about 50%, and 2)assessing the effectiveness of HQPC in replacing fish meal. In embodiments, optimizing soy, oil seed, and DDGS pretreatment and conversion conditions may be carried out to improve the performance and robustness of the microbes, test the resultant grower feeds for a range of commercially important fishes, validate process costs and energy requirements, and complete steps for scale-up and commercialization. In embodiments, the HQPC of the present disclosure may be able to replace at least 50% of fish meal, while providing increased growth rates and conversion efficiencies. Production costs should be less than commercial soy protein concentrate (SPC) and substantially less than fish meal.

After extrusion pretreatment, cellulose-deconstructing enzymes may be evaluated to generate sugars, which microbes of the present disclosure may convert to protein and gum. In embodiments, sequential omission of these enzymes and evaluation of co-culturing with cellulolytic microbes may be used. Ethanol may be evaluated to precipitate the gum and improve centrifugal recovery of the HQPC. After drying, the HQPC may be incorporated into practical diet formulations. In embodiments, test grower diets may be formulated (with mineral and vitamin premixes) and comparisons to a fish-meal control and commercial SPC (SPC is distinctly different from soybean meal, as it contains traces of oligopolysaccharides and antigenic substances glycinin and b-conglycinin) diets in feeding trials with a commercially important fish, e.g., yellow perch or rainbow trout, may be performed. Performance (e.g., growth, feed conversion, protein efficiency), viscera characteristics, and intestinal histology may be examined to assess fish responses.

In other embodiments, optimizing the HQPC/lipid production process by determining optimum pretreatment and conversion conditions while minimizing process inputs, improving the performance and robustness of the microbe, testing the resultant grower feeds for a range of commercially important fishes, validating process costs and energy requirements, and completing initial steps for scale-up and commercialization may be carried out.

In the past few years, a handful of facilities have installed a dry mill capability that removed corn hulls and germ prior to the ethanol production process. This dry fractionation process yields a DDGS with up to 42% protein (hereafter referred to as dryfrac DDGS). In embodiments, conventional and dryfrac DDGS under conditions previously determined to rapidly generate a sufficient amount of high protein DDGS (HP-DDGS) for use in perch feeding trials may be compared. In embodiments, careful monitoring of the performance of this conversion (via chemical composition changes) is carried out and parameters with the greatest impact on HP-DDGS quality identified. In some embodiments, low oil DDGS may be used as a substrate for conversion, where such low oil DDGS has the higher protein level than conventional DDGS. In a related aspect, low oil DDGS increase growth rates of A. pullulans compared to conventional DDGS.

Several groups are evaluating partial replacement of fish-meal with plant derived proteins, such as soybean meal and DDGS. However, the lower protein content, inadequate amino acid balance, and presence of anti-nutritional factors have limited the replacement levels to 20-40%. Preliminary growth trials indicate that no current DDGS or SPC-based diets provide performance similar to fish-meal control diets. Several deficiencies have been identified among commercially produced DDGS and SPCs, principally in protein and amino acid composition, which impart variability in growth performance and fish composition. However, HP-DDGS and HQSPC diets as disclosed herein containing nutritional supplements (formulated to meet or exceed all requirements) have provided growth results that are similar to or exceed fish-meal controls. Thus, the processes as disclosed herein and products developed therefrom provide a higher quality HQSPC or HP-DDGS (relative to nutritional requirements) and support growth performance equivalent to or better than diets containing fish meal.

Fish that can be fed the fish feed composition of the present disclosure include, but are not limited to, Siberian sturgeon, Sterlet sturgeon, Starry sturgeon, White sturgeon, Arapaima, Japanese eel, American eel, Short-finned eel, Long-finned eel, European eel, Chanos chanos, Milkfish, Bluegill sunfish, Green sunfish, White crappie, Black crappie, Asp. Catla, Goldfish, Crucian carp, Mud carp, Mrigal carp, Grass carp, Common carp, Silver carp, Bighead carp, Orangefin labeo, Roho labeo, Hoven's carp, Wuchang bream, Black carp, Golden shiner, Nilem carp, White amur bream, Thai silver barb, Java, Roach, Tench, Pond loach, Bocachico, Dorada, Cachama, Cachama Blanca, Paco, Black bullhead, Channel catfish, Bagrid catfish, Blue catfish, Wels catfish, Pangasius (Swai, Tra, Basa) catfish, Striped catfish, Mudfish, Philippine catfish, Hong Kong catfish, North African catfish, Bighead catfish, Sampa, South American catfish, Atipa, Northern pike, Ayu sweetfish, Vendace, Whitefish, Pink salmon, Chum salmon, Coho salmon, Masu salmon, Rainbow trout, Sockeye salmon, Chinook salmon, Atlantic salmon, Sea trout, Arctic char, Brook trout, Lake trout, Atlantic doe, Pejerrey, Lai, Common snook, Barramundi/Asian sea bass, Nile perch, Murray cod, Golden perch, Stripped bass, White bass, European seabass, Hong Kong grouper, Areolate grouper, Greasy grouper, Spotted coralgrouper, Silver perch, White perch, Jade perch, Largemouth bass, Smallmouth bass, European perch, Zander (Pike-perch), Yellow Perch, Sauger, Walleye, Bluefish, Greater amberjack, Japanese amberjack, Snubnose pompano, Florida pompano, Palometa pompano, Japanese jack mackeral, cobia, Mangrove red snapper, Yellowtail snapper, Dark seabream, White seabream, Crimson seabream, Red seabream, Red porgy, Goldlined seabream, Gilthead seabream, Red drum, Green terror, Blackbelt cichlid, Jaguar guapote, Mexican mojarra, Pearlspot, three spotted tilapia, Blue tilapia, Longfin tilapia, Mozambique tilapia, Nile tilapia, Tilapia, Wami tilapia, Blackchin tilapia, Redbreast tilapia, Redbelly tilapia, Golden grey mullet, Largescale mullet, gold-spot mullet, Thinlip grey mullet, Leaping mullet, Tade mullet, Flathead grey mullet, White mullet, Lebranche mullet, Pacific fat sleeper, marble goby, White-spotted spinefoot, Goldlined spinefoot, Marbled spinefoot, Southern bluefin tuna, Northern bluefin tuna, Climbing perch, Snakeskin gourami, Kissing gourami, Giant gourami, Snakehead, Indonesian snakehead, Spotted snakehead, Striped snakehead, turboi, Bastard halibut (Japanese flounder), Summer Flounder, Southern flounder, Winter flounder, Atlantic Halibut, Greenback flounder, Common sole, and combinations thereof.

It will be appreciated by the skilled person that the fish feed composition of the present disclosure may be used as a convenient carrier for pharmaceutically active substances.

The fish feed composition according to present disclosure may be provided as a liquid, pourable emulsion, or in the form of a paste, or in a dry form, for example as an extrudate, granulate, a powder, or as flakes. When the fish feed composition is provided as an emulsion, a lipid-in-water emulsion, it is may be in a relatively concentrated form. Such a concentrated emulsion form may also be referred to as a pre-emulsion as it may be diluted in one or more steps in an aqueous medium to provide the final enrichment medium for the organisms.

In embodiments, cellulosic-containing starting material for the microbial-based process as disclosed is corn. Corn is about two-thirds starch, which is converted during a fermentation and distilling process into ethanol and carbon dioxide. The remaining nutrients or fermentation products may result in condensed distiller's solubles or distiller's grains such as DDGS, which can be used in feed products. In general, the process involves an initial preparation step of dry milling or grinding of the corn. The processed corn is then subject to hydrolysis and enzymes added to break down the principal starch component in a saccharification step. The following step of fermentation is allowed to proceed upon addition of a microorganisms (e.g., yeast) provided in accordance with an embodiment of the disclosure to produce gaseous products such as carbon dioxide. The fermentation is conducted for the production of ethanol which may be distilled from the fermentation broth. The remainder of the fermentation medium may then be dried to produce fermentation products including DDGS. This step usually includes a solid/liquid separation process by centrifugation wherein a solid phase component may be collected. Other methods including filtration and spray dry techniques may be employed to effect such separation. The liquid phase components may be subjected further afterwards to an evaporation step that can concentrate soluble coproducts, such as sugars, glycerol and amino acids, into a material called syrup or condensed corn solubles (CCS). The CCS may then be recombined with the solid phase component to be dried as incubation products (DDGS). It shall be understood that the subject compositions and may be applied to new or already existing ethanol plants based on dry milling to provide an integrated ethanol production process that also generates fermentation products with increased value.

In embodiments, incubation products produced according to the present disclosure have a higher commercial value than the conventional fermentation products. For example, the incubation products may include enhanced dried solids with improved amino acid and micronutrient content. A “golden colored” product can be thus provided which generally indicates higher amino acid digestibility compared to darker colored HQSP. For example, a light-colored HQSP may be produced with an increased lysine concentration in accordance with embodiments herein compared to relatively darker colored products with generally less nutritional value. The color of the products may be an important factor or indicator in the assessing the quality and nutrient digestibility of the fermentation products or HQSP. Color is used as an indicator of exposure to excess heat during drying causing caramelization and Maillard reactions of the free amino groups and sugars, reducing the quality of some amino acids.

The basic steps in a dry mill or grind ethanol manufacturing process may be described as follows: milling or grinding of corn or other grain product, saccharification, fermentation, and distillation. For example, selected whole corn kernels may be milled or ground with typically either hammer mills or roller mills. The particle size can influence cooking hydration and subsequent enzymatic conversion. The milled or group corn can be then mixed with water to make a mash that is cooked and cooled. It may be useful to include enzymes during the initial steps of this conversion to decrease the viscosity of the gelatinized starch. The mixture may be then transferred to saccharification reactors, maintained at selected temperatures such as 140° F. where the starch is converted by addition of saccharifying enzymes to fermentable sugars such as glucose or maltose. the converted mash can be cooled to desired temperatures such as 84° F., and fed to fermentation reactors where fermentable sugars are converted to carbon dioxide by the use of selected strains of microbes provided in accordance with the disclosure that results in more nutritional fermentation products compared to more traditional ingredients such as Saccharomyces yeasts. The resulting product may be flashed to separate out carbon dioxide and the resulting liquid may be fed to a recovery system consisting of distillation columns and a stripping column. The ethanol stream may be directed to a molecular sieve where remaining water is removed using adsorption technology. Purified ethanol, denatured with a small amount of gasoline, may produce fuel grade ethanol. Another product may be produced by further purifying the initial distillate ethanol to remove impurities, resulting in about 99.95% ethanol for non-fuel uses.

The whole stillage may be withdrawn from the bottom of the distillation unit and centrifuged to produce distiller's wet grains (DWG) and thin stillage (liquids). The DWG may leave the centrifuge at 55-65% moisture, and may either be sold wet as cattle feed or dried as enhanced fermentation products provided in accordance with the disclosure. These products include an enhanced end product that may be referred to herein as distiller's dried grains (DDG). Using an evaporator, the thin stillage (liquid) may be concentrated to form distiller's solubles, which may be added back to and combined with a distiller's grains process stream and dried. This combined product in accordance with embodiments of the disclosure may be marketed as an enhanced fermentation product having increased amino acid and micronutrient content. It shall be understood that various concepts of the disclosure may be applied to other fermentation processes known in the field other than those illustrated herein.

Another aspect of the present invention is directed towards complete fish meal compositions with an enhanced concentration of nutrients which includes microorganisms characterized by an enhanced concentration of nutrients such as, but not limited to, fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin, silicon, and combinations thereof.

In an incubation process of the present disclosure, a carbon source may be hydrolyzed to its component sugars by microorganisms to produce alcohol and other gaseous products. Gaseous product includes carbon dioxide and alcohol includes ethanol. the incubation products obtained after the incubation process are typically of higher commercial value. In embodiments, the incubation products contain microorganisms that have enhanced nutrient content than those products deficient in the microorganisms. The microorganisms may be present in an incubation system, the incubation broth and/or incubation biomass. the incubation broth and/or biomass may be dried (e.g., spray-dried), to produce the incubation products with an enhanced content of the nutritional contents.

For example, the spent, dried solids recovered following the incubation process are enhanced in accordance with the disclosure. These incubation products are generally non-toxic, biodegradable, readily available, inexpensive, and rich in nutrients. The choice of microorganism and the incubation conditions are important to produce a low toxicity or non-toxic incubation product for use as a feed or nutritional supplement. While glucose is the major sugar produced from the hydrolysis of the starch from grains, it is not the only sugar produced in carbohydrates generally. Unlike the SPC or DDG produced from the traditional dry mill ethanol production process, which contains a large amount of non-starch carbohydrates (e.g., as much as 35% percent of cellulose and arabinoxylans-measured as neutral detergent fiber, by dry weight), the subject nutrient enriched incubation products produced by enzymatic hydrolysis of the non-starch carbohydrates are more palatable and digestible to the non-ruminant.

The nutrient enriched incubation product of this disclosure may have a nutrient content of from at least about 1% to about 95% by weight. The nutrient convent is preferably in the range of at least about 10%-20%, 20%-30%, 30%-40%, 40%-50%, 50%-60%, 60%-70%, and 70%-80% by weight. The available nutrient content may depend upon the animal to which it is fed and the context of the remainder of the diet, and stage in the animal life cycle. For instance, beef cattle require less histidine than lactating cows. Selection of suitable nutrient content for feeding animals is well known to those skilled in the art.

The incubation products may be prepared as a spray-dried biomass product. Optionally, the biomass may be separated by known methods, such as centrifugation, filtration, separation, decanting, a combination of separation and decanting, ultrafiltration or microfiltration. The biomass incubation products may be further treated to facilitate rumen bypass. In embodiments, the biomass product may be separated from the incubation medium. spray-dried, and optionally treated to modulate rumen bypass, and added to feed as a nutritional source. In addition to producing nutritionally enriched incubation products in an incubation process containing microorganisms, the nutritionally enriched incubation products may also be produced in transgenic plant systems. Methods for producing transgenic plant systems are known in the art. Alternatively, where the microorganism host excretes the nutritional contents, the nutritionally-enriched broth may be separated from the biomass produced by the incubation and the clarified broth may be used as an animal feed ingredient, e.g., either in liquid form or in spray dried form.

The incubation products obtained after the incubation process using microorganisms may be used as an animal feed or as food supplement for humans. The incubation product includes at least one ingredient that has an enhanced nutritional content that is derived from a non-animal source (e.g., a bacteria, yeast, and/or plant). In particular, the incubation products are rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. In embodiments, the peptides contain at least one essential amino acid. In other embodiments, the essential amino acids are encapsulated inside a subject modified microorganism used in an incubation reaction. In embodiments, the essential amino acids are contained in heterologous polypeptides expressed by the microorganism. Where desired, the heterologous peptides are expressed and stored in the inclusion bodies in a suitable microorganism (e.g., fungi).

In embodiments, the incubation products have a high nutritional content. As a result, a higher percentage of the incubation products may be used in a complete animal feed. In embodiments, the feed composition comprises at least about 15% of incubation product by weight. In a complete feed, or diet, this material will be fed with other materials. Depending upon the nutritional content of the other materials, and/or the nutritional requirements of the animal to which the feed is provided, the modified incubation products may range from 15% of the feed to 100% of the feed. In embodiments, the subject incubation products may provide lower percentage blending due to high nutrient content. In other embodiments, the subject incubation products may provide very high fraction feeding, e.g. over 75%. In suitable embodiments, the feed composition comprises at least about 20%, at least about 25%, at least about 30%, at least about 35%, at least about 40%, at least about 45%, at least about 50%, at least about 60%, at least about 70%, or at least about 75% of the subject incubation products. Commonly, the feed composition comprises at least about 20% of incubation product by weight. More commonly, the feed composition comprises at least about 15-25%, 25-20%, 20-25%, 30%-40%, 40%-50%, 50%-60%, or 60%-70% by weight of incubation product. Where desired, the subject incubation products may be used as a sole source of feed.

The complete fish meal compositions may be enhanced amino acid content with regard to one or more essential amino acids for a variety of purposes, e.g., for weight increase and overall improvement of the animal's health. The complete fish meal compositions may have enhanced amino acid content because of the presence of free amino acids and/or the presence of proteins or peptides including an essential amino acid, in the incubation products. Essential amino acids may include histidine, lysine, methionine, phenylalanine, threonine, taurine (sulfonic acid), isoleucine, and/or tryptophan, which may be present in the complete animal feed as a free amino acid or as part of a protein or peptide that is rich in the selected amino acid. At least one essential amino acid-rich peptide or protein may have at least 1% essential amino acid residues per total amino acid residues in the peptide or protein, at least 5% essential amino acid residues per total amino acid residues in the peptide or protein, or at least 10% essential amino acid residues per total amino acid residues in the protein. By feeding a diet balanced in nutrients to animals, maximum use is made of the nutritional content, requiring less feed to achieve comparable rates of growth, milk production, or a reduction in the nutrients present in the excreta reducing bioburden of the wastes.

A complete fish meal composition with an enhanced content of an essential amino acid, may have an essential amino acid content (including free essential amino acid and essential amino acid present in a protein or peptide) of at least 2.0 wt % relative to the weight of the crude protein and total amino acid content, and more suitably at least 5.0 wt % relative to the weight of the crude protein and total amino acid content. The complete fish meal composition includes other nutrients derived from microorganisms including but not limited to, fats, fatty acids, lipids such as phospholipid, vitamins, carbohydrates, sterols, enzymes, and trace minerals.

The complete fish meal composition may include complete feed form composition, concentrate form composition, blender form composition, and base form composition. If the composition is in the form of a complete feed, the percent nutrient level, where the nutrients are obtained from the microorganism in an incubation product, which may be about 10 to about 25 percent, more suitably about 14 to about 24 percent; whereas, if the composition is in the form of a concentrate, the nutrient level may be about 30 to about 50 percent, more suitably about 32 to about 48 percent. If the composition is in the form of a blender, the nutrient level in the composition may be about 20 to about 30 percent, more suitably about 24 to about 26 percent; and if the composition is in the form of a base mix, the nutrient level in the composition may be about 55 to about 65 percent. Unless otherwise stated herein, percentages are stated on a weight percent basis. If the HQPC is high in a single nutrient, e.g., Lys, it will be used as a supplement at a low rate; if it is balanced in amino acids and Vitamins, e.g., vitamin A and E, it will be a more complete feed and will be fed at a higher rate and supplemented with a low protein, low nutrient feed stock, like corn stover.

The fish meal composition may include a peptide or a crude protein fraction present in an incubation product having an essential acid content of at least about 2%. In embodiments, a peptide or crude protein fraction may have an essential amino acid content of at least about 3%, at least about 5%, at least about 10%, at least about 15%, at least about 20%, at least about 30%, at least about 40%, and in embodiments, at least about 50%. In embodiments, the peptide may be 100% essential amino acids. Commonly, the fish meal composition may include a peptide or crude protein fraction present in an incubation product having an essential amino acid content of up to about 10%. More commonly, the fish meal composition may include a peptide or a crude protein fraction present in an incubation product having an essential amino acid content of about 2-10%, 3.0-8.0%, or 4.0-6.0%.

The fish meal composition may include a peptide or a crude protein fraction present in an incubation product having a lysine content of at least about 2%. In embodiments, the peptide or crude protein fraction may have a lysine content of at least about 3%, at least about 5%, at least about 10%, at least about 15%, at least about 20%, at least about 30%, at least about 40%, and in embodiments, at least about 50%. Typically, the fish meal composition may include the peptide or crude protein fraction having a lysine content of up to about 10%. Where desired, the fish meal composition may include the peptide or a crude protein fraction having a lysine content of about 2-10%, 3.0-8.0%, or 4.0-6.0%.

The fish meal composition may include nutrients in the incubation product from about 1 g/Kg dry solids to 900 g/Kg dry solids. In embodiments, the nutrients in a fish meal composition may be present to at least about 2 g/Kg dry solids, 5 g/Kg dry solids, 10 g/Kg dry solids, 50 g/Kg dry solids, 100 g/Kg dry solids, 200 g/Kg dry solids, and about 300 g/Kg dry solids. In embodiments, the nutrients may be present to at least about 400 g/Kg dry solids, at least about 500 g/Kg dry solids, at least about 600 g/Kg dry solids, at least about 700 g/Kg dry solids, at least about 800 g/Kg dry solids and/or at least about 900 g/Kg dry solids.

The fish meal composition may include an essential amino acid or a peptide containing at least one essential amino acid present in an incubation product having a content of about 1 g/Kg dry solids to 900 g/Kg dry solids. In embodiments, the essential amino acid or a peptide containing at least one essential amino acid in a fish meal composition may be present to at least about 2 g/Kg dry solids, 5 g/Kg dry solids, 10 g/Kg dry solids, 50 g/Kg dry solids, 100 g/Kg dry solids, 200 g/Kg dry solids, and about 300 g/Kg dry solids. In embodiments, the essential amino acid or a peptide containing at least one essential amino acid may be present to at least about 400 g/Kg dry solids, at least about 500 g/Kg dry solids, at least about 600 g/Kg dry solids, at least about 700 g/Kg dry solids, at least about 800 g/Kg dry solids and/or at least about 900 g/Kg dry solids.

The complete fish meal composition may contain a nutrient enriched incubation product in the form of a biomass formed incubation and at least one additional nutrient component. In another example, the fish meal composition contains a nutrient enriched incubation product that is dissolved and suspended from an incubation broth formed during incubation and at least one additional nutrient component. In a further embodiment, the fish meal composition has a crude protein fraction that includes at least one essential amino acid-rich protein. The fish meal composition may be formulated to deliver an improved balance of essential amino acids.

For compositions comprising DDGS, the complete composition form may contain one or more ingredients such as wheat middlings (“wheat midds”), corn, soybean meal, corn gluten meal, distiller's grains or distiller's grains with solubles, salt, macro-minerals, trace minerals and vitamins. Other potential ingredients may commonly include, but not be limited to sunflower meal, malt sprouts and soybean hulls. The blender form composition may contain wheat middlings, corn gluten meal, distiller's grains or distiller's grains with solubles, salt, macro-minerals, trace minerals and vitamins. Alternative ingredients would commonly include, but not be limited to, corn, soybean meal, sunflower meal, cottonseed meal, malt sprouts and soybean hulls. The base form composition may contain wheat middlings, corn gluten meal, and distiller's grains or distiller's grains with solubles. Alternative ingredients would commonly include, but are not limited to, soybean meal, sunflower meal, malt sprouts, macro-minerals, trace minerals and vitamins.

Highly unsaturated fatty acids (HUFAs) in microorganisms, when exposed to oxidizing conditions may be converted to less desirable unsaturated fatty acids or to saturated fatty acids. However, saturation of omega-3 HUFAs may be reduced or prevented by the introduction of synthetic antioxidants or naturally-occurring antioxidants, such as beta-carotene, vitamin E and vitamin C, into the feed. Synthetic antioxidants, such as BHT, BHA, TBHQ or ethoxyquin, or natural antioxidants such as tocopherols, may be incorporated into the food or feed products by adding them to the products, or they may be incorporated by in situ production in a suitable organisms. The amount of antioxidants incorporated in this manner depends, for example, on subsequent use requirements, such as product formulation, packaging methods, and desired shelf life.

Incubation products or complete fish meal containing the incubation products of the present disclosure, may also be utilized as a nutritional supplement for human consumption if the process begins with human grade input materials, and human food quality standards are observed through out the process. Incubation product or the complete feed as disclosed herein is high in nutritional content. Nutrients such as, protein and fiber are associated with healthy diets. Recipes may be developed to utilize incubation product or the complete feed of the disclosure in foods such as cereal, crackers, pies, cookies, cakes, pizza crust, summer sausage, meat balls, shakes, and in any forms of edible food. Another choice may be to develop the incubation product or the complete feed of the disclosure into snacks or a snack bar, similar to granola bar that could be easily eaten, convenient to distribute. A snack bar may include protein, fiber, germ, vitamins, minerals, from the grain, as well as nutraceuticals such as glucosamine, HUFAs, or co-factors, such as Vitamin Q-10.

The fish meal comprising the subject incubation products may be further supplemented with flavors. The choice of a particular flavor will depend on the animal to which the feed is provided. The flavors and aromas, both natural and artificial, may be used in making feeds more acceptable and palatable. These supplementations may blend well with all ingredients and may be available as a liquid or dry product form. Suitable flavors, attractants, and aromas to be supplemented in the animal feeds include but not limited to fish pheromones, fenugreek, banana, cherry, rosemary, cumin, carrot, peppermint oregano, vanilla, anise, plus rum, maple, caramel, citrus oils, ethyl butyrate, menthol, apple, cinnamon, any natural or artificial combinations thereof. The flavors and aromas may be interchanged between different animals. Similarly, a variety of fruit flavors, artificial or natural may be added to food supplements comprising the subject incubation products for human consumption.

The shelf-life of the incubation product or the complete feed of the present disclosure may typically be longer than the shelf life of an incubation product that is deficient in the microorganism. The shelf-life may depend on factors such as, the moisture content of the product, how much air can flow through the feed mass, the environmental conditions and the use of preservatives. A preservative may be added to the complete feed to increase the shelf life to weeks and months. Other methods to increase shelf life include management similar to silage management such as mixing with other feeds and packing, covering with plastic or bagging. Cool conditions, preservatives and excluding air from the feed mass all extend shelf life of wet co-products. The complete feed can be stored in bunkers or silo bags. Drying the wet incubation product or complete feed may also increase the product's shelf life and improve consistency and quality.

The complete feed of the present disclosure may be stored for long period of time. The shelf life may be extended by ensiling, adding preservatives such as organic acids, or blending with other feeds such as soy hulls. Commodity bins or bulk storage sheds may be used for storing the complete feeds.

As used herein, “room temperature” is about 25° C. under standard pressure.

The following examples are illustrative and are not intended to limit the scope of the disclosed subject matter.

### EXAMPLES

### Example 1

### Production of 1st Generation HP-DDGS

In a pretreatment evaluation, DDG was extruded in a single screw extruder (BRABENDER PLASTI-CORDER EXTRUDER MODEL PL2000, Hackensack, N.J.) with a barrel length to screw diameter of 1:20 and a compression ratio of 3:1. DDG was adjusted to 25-30% moisture, the extrusion temperature was set at 175° C., and screw speed was set at 50 rpm, providing a shearing effect against the ridged channels on both sides of the barrel. This was referred to as extrusion method 1. These selected levels of temperature, screw speed and moisture were based on optimized conditions defined previously for defatted soybean meal.

Extruded DDG (50 Kg) was then mixed with 450 L water to achieve a solid loading rate of 10% in a 600 L bioreactor. The pH was adjusted to 5 and the slurry was heated. After cooling the slurry was saccharified using a cocktail of enzymes. The temperature was then reduced, the pH was adjusted to 3.0 (to optimize cell growth), and the slurry was inoculated with 2% (v/v) of a 24 h culture. The slurry was then aerated in a submerged state for 96 h. during incubation, samples were removed at 12-24 h intervals for pH, HPLC (sugars), and culture purity analysis. Following incubation the converted slurry was subjected to ethanol precipitation and centrifugation to recover the protein and microbial biomass (HP-DDGS). While not being bound by theory, the presence of a precipitating gum improves the efficiency of centrifugation in recovering suspended solids. Approximately 33.3 Kg of solids were recovered, with a protein concentration of 43.43% on a dry basis. This HP-DDGS (referred to as Submerged WT28) was used in fish feeding trials.

### Solid State Trials

Separate trials were conducted with non-extruded DDGS (trial PAT 2.3) vs non-extruded DDG (trial PAT 2.4). Both feedstocks (3.5 Kg) were mixed with water to achieve a moisture content of 50%, the pH was adjusted to 3-3.5, and 2 ml of a 10-2 stock solution of a commercial antibiotic was added to prevent bacterial contamination. A 6.25% (v/v) inoculum of a 24-48 h microbe culture was mixed into the solid pulp. These materials were then placed into separate 16 cm diameter by 76 cm tall tubes that were fitted with false bottoms to permit an upward flow of humidified air. The tubes were incubated statically for 168 h at room temperature. Following incubation the solids were removed, dried, and analyzed for protein content. The DDGS sample (PAT 2.3) was 39.75% protein and the DDG (PAT 2.4) was 41.28% protein. Thus the protein levels were lightly lower in the solid state trials with non-extruded feedstocks compared to the 1st generation product. While not being bound by theory, it was though that this was primarily due to the “washing” effect in the prior submerged conversion process. HP-DDGS products were also tested in the fish feeding trials.

### Comparison of DDG Pretreatment in a Submerged Process

Using non-extruded DDGS and non-extruded DDG as controls, we next evaluated several additional pretreatments on DDG using the submerged process. A dilute acid pretreatment was performed using 1% H₂SO₄ solution at 121° C. for 20 minutes. Hot water cook pretreatment was performed at 160° C. for 20 min. Extrusion of 25-30% moisture DDG was conducted as described above (175° C. and 50 rpm, extrusion method 1). A refined commercial DDGS (StillPro) that contains reduced fiber levels was also tested.

For conversion, pretreated feedstocks were mixed with water to achieve a solid loading rate of 10% in a 5 L New Brunswick Bioflo 3 bioreactor (3-4 L working volume), at a pH of 5. After autoclaving and cooling, the slurry was saccharified for 24 h. The temperature was then reduced to 30° C., the pH was either left at 5 or reduced to 3, and the slurry was inoculated with 2% (v/v) of a 24 h culture. The slurry was then aerated for 120 h. During incubation, samples were removed at 6-12 h intervals. Samples were subjected to HPLC analysis for carbohydrates and hemocytometer counts to assess microbial populations. Samples were also subjected to ethanol precipitation and centrifugation to separate the protein and microbial biomass (HP-DDGS).

### Evaluate the Performance of HP-DDGS as a Fish Meal Replacement in Perch Feeds

Products derived from the above processes were analyzed for nutritional competencies in view of requirements of targeted species, especially focusing on yellow perch. Samples were subjected to chemical analyses (proximate analysis, fiber, insoluble carbohydrates, amino acids, fatty acids, and minerals) prior to feed formulation.

### Experiment Design Summary

The feeding trial was conducted in a recirculating aquaculture system (RAS). Replication of four experimental units (20 fish/110 L tank) per treatment was used in the feeding trial which lasted 112 days. A heat pump was used to maintain the optimal temperature (23.6° C.) for yellow perch growth. Water quality (e.g., dissolved oxygen, pH, temperature, ammonia and nitrite) was monitored daily.

Experimental diets were delivered according to fish size (˜5 g starting weight), split into two daily feedings of 60% daily ration in the morning and 40% daily ration in the evening. Growth performance was determined by total mass measurements taken every four weeks. Rations were adjusted in accordance with gains, which allowed for satiation with respect to feeding and to reduce waste streams. Consumption was assessed by counting uneaten pellets remaining in the tank 30 minutes after feeding and adjusting to 90% consumption of fed pellets. Survival, weight gain, growth rate, health indices, feed conversion, protein and energy digestibilities, and protein efficiency were compared among treatment groups.

Statistical analyses of diets and feeding trial responses was completed with an a priori α=0.05. An analysis of performance parameters among treatments was done with appropriate analysis of variance or covariance (Proc Mixed) and post hoc multiple comparisons, as needed.

### Feed Preparation

Complete practical diets were formulated using DDGS or converted DDGS in accordance with known nutrient requirements for yellow perch (e.g., 45% protein, 9% lipid) in a factorial design. Basal mineral and vitamin premixes for plant-based diets were used to meet micro-nutrient requirements. All feeding trials including a fish meal-based control diet and diets containing a range of DDGS products, both commercial and experimental.

Seven test protein ingredients including experimental DDGS products, commercial DDGS, and a menhaden fish meal control were used in diet formulations (Table 1). Diets were formulated to be isonitrogenous, and isolipidic by adjusting wheat gluten, wheat flour, cellufil, menhaden and corn oils. Targeted diet proximate compositions (dmb) were 45% protein, 9% lipid, and protein to energy ratios (PE) of approximately 27 g protein/MJ GE (Table 2). All diets were formulated as compound practical diets, which included vitamin and mineral supplements as well as palatability and pellet quality augmentations. A completely randomized nested design was implemented wherein each of the DDGS diets were duplicated and supplemented with taurine, methionine, histidine, and arginine to meet or exceed known yellow perch requirements.

TABLE 1
Base ingredients incorporated in the feeding trials

| Ingredient | Description |
|---|---|
| Fish Meal | Control diet |
| Raw DDG | DDG from Dakota Ethanol (Wentworth, SD) |
| Still Pro 50 | Mechanically fractionated (post-fermentation) |
| | DDGS |
| Novita NovaMeal | Solvent extracted (hexane) DDGS |
| | (experimental product) |
| Converted Wet Cake | Extruded, saccharified wet cake microbially |
| (WT28) | converted in submerged reactor. |
| Converted Wet Cake | Non-extruded, non-saccharified wet cake |
| (PAT 2.4) | microbially converted in the tubular type |
| | solid state reactor. |
| Converted DDGS | Non-extruded, non-saccharified DDGS |
| (PAT 2.3) | microbially converted in the tubular type solid |
| | state reactor. |

Large particle ingredients were ground with a Fitzpatrick comminutor (Fitzpatrick Company, Elmhurst, Ill.) with 0.51 mm screen prior to dry blending. Dry ingredients were blended for 15 minutes using a Hobart HL200 mixer before water and oils were added and then blended for an additional 5 min. Feeds were then screw-pressed using a Hobart 4146 grinder with a 2.0 mm die and dried with a Despatch conveyor dryer at 210° F. Following drying, feeds were placed in frozen storage at −20° C., pending feeding. Approximately 7 kg of each diet were prepared, including 3.5 kg containing 1% (dry diet) chromic oxide for apparent digestibility determinations.

Chemical analyses of primary protein sources (Table 2) and feeds (Table 3) were completed by private labs. Analyses were completed only on the four basal diets because lysine and methionine were supplemented in known concentrations. Analyses were completed for crude protein (AOAC 2006, method 990.03), crude fat (AOAC 2006, method 990.03), crude fiber (AOAC 2006, method 978.10), moisture (AOAC 2006, method 934.01), and ash (AOAC, method 942.05) and amino acids (AOAC 2006, method 982.30 E (a,b,c)).

TABLE 2
Base ingredient compositional profile (dry weight basis)

| | | | | Converted | Converted | Converted |
|---|---|---|---|---|---|---|
| | | | | Wet Cake | Wet Cake | DDGS |
| Composition | Raw | Still Pro | | (Submerged) | (Solid state) | (Solid state) |
| (dry basis) | DDG | 50 | Novita | WT28 | PAT 2.4 | PAT 2.3 |
| Protein % | 31.93 | 49.41 | 34.36 | 43.43 | 39.75 | 41.38 |
| Fat % | 8.09 | 3.24 | 0.99 | 1.89 | 9.93 | 7.29 |
| Carbohydrate % | 48.38 | 39.50 | 53.19 | 41.09 | 38.68 | 39.38 |
| Fiber % | 5.85 | 3.58 | 6.73 | 8.33 | 6.67 | 9.31 |
| Ash % | 1.77 | 4.27 | 4.73 | 5.26 | 4.97 | 2.64 |
| Dry Matter | 0.66 | 0.963 | 0.99 | 0.99 | 0.96 | 0.96 |

TABLE 3
Predicted dietary proximates (g/100 g dmb, unless noted).

| | Diet Treatment | | | | | | |
|---|---|---|---|---|---|---|---|
| | FM | SP50 | WT28 | DDG | PAT 2.4 | PAT 2.3 | Novita |
| Protein (%) | 40.43 | 37.46 | 41.76 | 42.96 | 41.18 | 43.03 | 42.25 |
| Lipid (%) | 10.1 | 8.46 | 9.42 | 10 | 8.97 | 9.41 | 9.07 |
| Ash (%) | 32.5 | 42.4 | 36.2 | 36.5 | 37.2 | 33.1 | 38.2 |
| Gross energy (MJ/ | 7.93 | 5.31 | 5.09 | 3.39 | 5.55 | 6.44 | 2.69 |
| PE (g/MJ) | 8.99 | 6.37 | 7.51 | 7.17 | 7.14 | 7.99 | 7.85 |

### Feeding Trial Design

560 juvenile yellow perch (μ±SE, 4.13±0.64 g) were randomly stocked into 28 circular plastic tanks (110 L) within the RAS tanks. The initial tank mass (21 fish per tank, 86.78±2.94 g) was not significantly different (p=0.76) among tanks. After three days of system acclimation on the commercial diet, fish were introduced a graded mixture of the commercial diet and the specific treatment diet for four days and then fed 100% treatment diet for one week. On the start of the trial, the fish biomass per tank was weighed and visible health was monitored.

Fish were fed to satiation by hand twice daily, and feeding rates were modified according to fish weight by tank, observed growth rates, and feed consumption assessments. Consumption rates (%) were estimated from dividing the weight of uneaten from the total feed offered. The weight of uneaten feed was calculated from counting the number of uneaten pellets 30 min after feeding which corresponded with the time when pellets started to disintegrate and individual pellets would no longer be eaten or distinguished. This was chosen as the consumption method because of ease of implementation, and estimated consumption twice per week to correlate with the specific feeding period ration. Tank consumption estimates were performed twice a week and multiplied by rations fed to obtain feed consumption (g). Fish biomass by tank (+0.01 g) was measured every four weeks to monitor fish health and calculate growth performance.

Individual lengths (mm) and weights (+0.01 g) were also measured every four weeks on a randomly sampled fish from each treatment. Other performance variables measured were:

Feed conversion ratio (FCR: calculated as:

$$F C R = \frac{mass of feed consumed ( dry , g )}{growth ( wet , g )}$$

Protein conversion ratio (PER), calculated as:

$$P E R = \frac{growth ( wet , g )}{mass of protein consumed ( dry , g )}$$

Fulton-type condition factor (K); calculated as:

$$K = \frac{weight ( g )}{length {( mm )}^{3}} \times 100 , 000.$$

Specific growth rate (SGR); calculated as:

$$S G R = \frac{[ ln ( final wt ( g ) ) - ln ( start wt ( g ) ) ] \times 100}{n ( days )}$$

Protein and energy digestion of trial ingredients were estimated using a chromic oxide (CrO₃) marker within the feed. Fecal material was collected via stripping and necropsy from the distal ⅓ of intestinal tract at the conclusion of the feeding trial.

### Results and Discussion

The composition of the HP-DDGS was determined and is shown in Table 4.

TABLE 4
Comparison of DDG microbial pretreatments
with in a submerged process

| | | | Final Protein |
|---|---|---|---|
| Feedstock | Pretreatment | Incubation pH | (%, dmb) |
| DDGS | Non extruded | 5 | 45.75 |
| DDG | Non extruded | 5 | 38-42 |
| DDG | Dilute acid | 5 | 38.5 |
| DDG | Hot H20 cook | 5 | 48 |
| DDG | Hot H20 cook | 3 | 43 |
| DDG | Extrusion 1 | 5 | 38-41 |
| DDG | Extrusion 1 | 3 | 46.50 |
| DDG | Extrusion 2 | 3 | 49.90 |
| StillPro DDGS | Non extruded | 3 | 64.44 |

Non-extruded DDGS resulted in a 45.75% protein product in the submerged trial, compared to ˜40% protein in the solid state trials, again, while not being bound by theory, may be due to an added “washing” effect in the submerged trial. However in the non-extruded DDG trial the final protein levels were similar: 38-42% in the submerged trial (Table 4) vs ˜41% in the prior solid state trial. These protein levels were also comparable to the 41-43% protein of the extruded DDG in the 1^{st} generation product, suggesting that extrusion method 1 provided no significant benefit. Of the other pretreatments tested, dilute acid did not improve protein concentrations. However the hot water cook pretreatment showed a significant improvement.

### Comparison of Cellulolytic Fungi on Extruded DDG (Method 1) in a Submerged Process

To establish whether expensive cellulase enzymes could be replaced by using cellulolytic fungi we tested DDG processed via extrusion method 1 using the same protocol as above, except that the cellulase enzymes and saccharification step were omitted. The results demonstrate protein levels of 36-45.6% when cellulase enzymes were replaced by specific cellulolytic fungi compared to the 38-42% protein levels observed when cellulase enzymes were used with a non-cellulolytic strain.

### Growth Trial Results

### Feed Nutrition

The predicted diet composition of 45% protein is shown in Table 5. All diets were supplemented with arginine, lysine, histidine, methionine, and taurine to meet, or exceed, minimum yellow perch requirements.

TABLE 5
Calculated diet compositions used in the feeding trial.

| | Fish | Raw Wet | Still Pro | | Submerged | SSFPAT | SSF PAT |
|---|---|---|---|---|---|---|---|
| Diet | Meal | Cake | 50 | Novita | WT28 | 2.4 | 2.3 |
| Protein % | 45.00 | 45.00 | 45.00 | 45.00 | 45.00 | 45.00 | 45.00 |
| Digestible | 40.57 | 41.08 | 38.39 | 40.20 | 39.94 | 40.27 | 39.89 |
| Lipid % | 9.00 | 9.00 | 9.00 | 9.00 | 9.00 | 9.00 | 9.00 |
| Fiber % | 0.97 | 2.82 | 1.68 | 2.95 | 2.90 | 2.66 | 3.24 |
| Ash % | 14.09 | 18.32 | 15.45 | 17.74 | 16.17 | 16.72 | 16.47 |
| Carbohydrate % | 13.46 | 40.10 | 21.25 | 28.91 | 22.75 | 23.03 | 22.81 |
| GE (MJ/kg) | 16.64 | 21.54 | 18.10 | 19.64 | 18.57 | 18.58 | 18.64 |
| Digestible GE | 15.02 | 19.03 | 15.56 | 17.12 | 16.29 | 16.44 | 16.26 |
| PE (g/MJ) | 27.03 | 20.88 | 24.84 | 22.90 | 24.21 | 24.21 | 24.13 |
| Digestible PE | 26.99 | 21.57 | 24.66 | 23.46 | 24.50 | 24.48 | 24.52 |

### Growth Performance

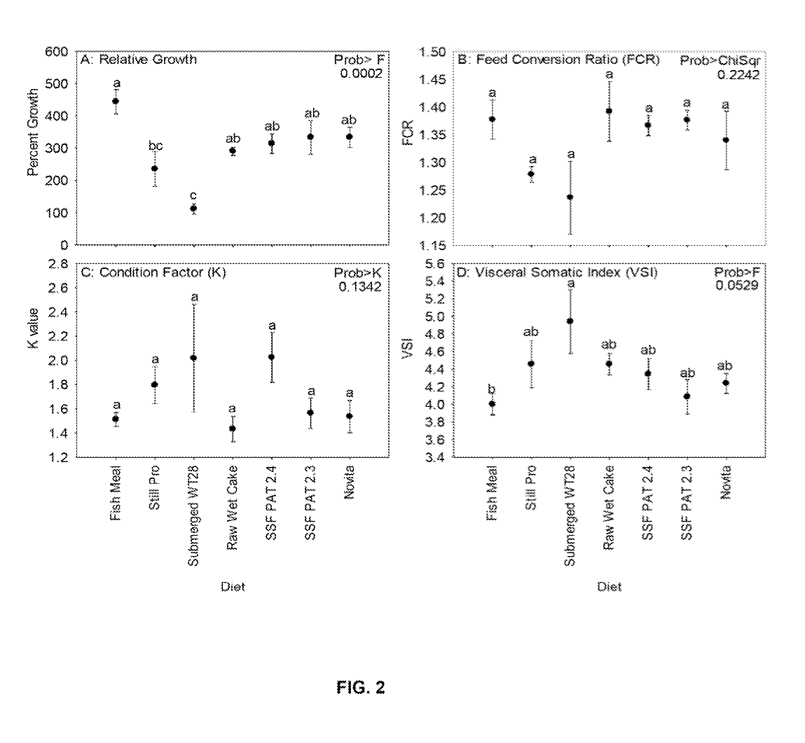

The growth trial metrics were analyzed following the Day 112 final sampling. Final relative growth is displayed in FIG. 2. The fish meal control showed the highest relative growth (443.53±37.73 g) while SSF PAT 2.3 (333.08±52.05 g; p=0.2059) and PAT 2.4 (313.86±40.44 g; p=0.3682) demonstrated similar performance to this reference diet. The submerged treatment (111.61±15.91 g) displayed the lowest relative growth performance and was significantly different from the fish meal control diet (p<0.0001).

Fish meal also produced a significantly higher tank biomass (678.90 g) than all other treatments. SSF PAT 2.4 (557.33 g) and SSF PAT 2.3 (542.65 g) produced the next highest tank biomass. Submerged WT28 (248.75 g), produced significantly lower biomass than all other treatments. The commercial corn-based diets, Still Pro 50 (485.53 g) and Novita (512.68 g), produced similar tank biomass.

SGR followed a similar performance trend with fish meal (2.01) outperforming all corn-based diets but was only significantly different from Submerged WT28 (0.88). Survival was significantly different between groups (p=0.3424). SSF PAT 2.4 and Still Pro 50 had the highest survival rates (90%) but were not significantly different from the other dietary treatments. Fulton's condition factor (K) was not significantly different between treatments (p=0.1324), but was highest for fish fed raw wet cake (1.39) and lowest for submerged WT28 (1.24). Feed conversion ratio (FCR) were not significantly different between diets (p=0.22). The results indicate that raw wet cake displayed the best FCR (1.43) (FIG. 2). SSF PAT 2.4 also produced the best FCR (1.37) for the experimental HP-DDG blends. Protein efficiency ratio (PER) was significantly different between treatments (p=0.028). PER was highest in fish meal (1.25) followed by raw wet cake (1.21), and was only significantly different from Submerged WT28 (0.79).

### Necropsy Variables

Upon completion of the trial, five fish per tank were euthanized and dissected to characterize fish health due to diet responses. There were significant differences in fish morphology and anatomy as a result of the experimental diets (Table 6).

TABLE 6
Summary (means ± standard error) of health indices (HSI, hepatosomatic;
VSI, visceral somatic; VFI, visceral fat; SSI, spleen somatic) at Day 112.

| Index (%) | Fish Meal | Still Pro 50 | Submerged WT28 | Raw Wet Cake | SSF PAT 2.4 | SSF PAT 2.3 | Novita |
|---|---|---|---|---|---|---|---|
| Fillet/body | 31.53 ± 1.33^{ab} | 33.24 ± 1.86^{a} | 26.50 ± 1.39^{b} | 29.49 ± 1.20^{ab} | 30.17 ± 1.489^{ab} | 31.10 ± 0.77^{ab} | 32.80 ± 0.90^{a} |
| weight | | | | | | | |
| HSI | 1.50 ± 0.06^{b} | 1.58 ± 0.08^{ab} | 1.68 ± 0.14^{ab} | 1.72 ± 0.04^{ab} | 1.63 ± 0.06^{ab} | 1.47 ± 0.06^{b} | 1.89 ± 0.06^{a} |
| VSI | 4.00 ± 0.12^{b} | 4.46 ± 0.27^{ab} | 4.94 ± 0.36^{a} | 4.46 ± 0.12^{ab} | 4.34 ± 0.18^{ab} | 4.09 ± 0.20^{ab} | 4.24 ± 0.11^{ab} |
| VFI | 4.42 ± 0.23^{a} | 3.85 ± 0.29^{a} | 3.41 ± 0.31^{a} | 3.63 ± 0.26^{a} | 4.14 ± 0.21^{a} | 3.79 ± 0.20ₐ | 3.46 ± 0.22^{a} |
| SSI | 0.058 ± 0.01^{a} | 0.058 ± 0.007^{a} | 0.059 ± 0.006^{a} | 0.058 ± 0.005^{a} | 0.065 ± 0.009^{a} | 0.089 ± 0.031^{a} | 0.048 ± 0.004^{a} |

No differences were observed for the visceral fat index (VFI) among treatments (p=0.051). The Submerged WT28 exhibited the lowest VFI (3.41). All of the solid-state fermentation diets (SSF PAT 2.4 and SSF PAT 2.3) produced fish which on average had a higher VFI than the commercial Still Pro 50 diet. Fat in the visceral cavity is considered an indication of poor health. In addition, excess lipids can affect the visual sense, odor of the final product and decrease the carcass yield.

Hepatosomatic index (HSI) was significantly different between diets (p=0.005). During necropsy, livers of some treatment fish seemed to have a pale color. A pale liver color has been found in other species that have been fed diets with essential fatty acid deficiencies. When fish are not utilizing lipids properly or there is imbalance of n-3/n-6 fatty acids. Submerged WT28 had a greater variance than the other diets with HSI's encompassing other treatments. No significant differences existed in spleen somatic (p=0.659) or visceral fat indices (p=0.051).

The production process has undergone significant changes, which have resulted in substantial reduction in product costs. A comparison of the mass balance can be seen in Table 7.

TABLE 7
Mass Balance for Generation 1 (Submerged)
and Generation 2 (Solid State) Process

| | | | | | Mois- | Mass |
|---|---|---|---|---|---|---|
| Process | Process | Process Stream | Mass Dry | | ture | Recov- |
| Generation | Step | Name | Basis | | Percent | ery |
| Gen 1 | Fill | Raw Materials | 50 | kg | 90% | |
| Pilot | Incubation | Process Slurry | 42 | kg | 90% | 80% |
| Scale | Incubation | Gases, Vapors | 8 | kg | 100% | |
| | Separation | Wet Grains | 33 | kg | 72% | 66% |
| | Separation | Thin Grains | 9 | kg | 97% | |
| | Drying | Vapor | | | 100% | |
| | Drying | Product | 33 | kg | 8% | 66% |
| Gen 2 | Fill | Raw Materials | 3.5 | kg | 50% | |
| Lab | Incubation | Process Slurry | 3.0 | kg | 50% | 86% |
| Scale | Incubation | Gases Vapors | 0.5 | kg | 100% | |
| | Drying | Vapor | | | 100% | |
| | Drying | Product | 3.0 | kg | 8% | 86% |

The Generation 1 data is from a 50 kg process run that produced 33 kg of product resulting in a 66% percent product yield. The loss of mass occurs both from the respiration losses and losses in the concentrate. The Generation 2 data is from a 3.5 kg process run that produced 3.0 kg of product resulting in an 86% product yield. The Generation 2 process results in a more efficient mass balance because it does not have the losses associated with the concentrate. The loss of non-protein components in the concentrate has given increased protein concentrations, but it is anticipated that further optimization of the solid-state process can mitigate this impact. It is anticipated that the product recovery will be further improved as the process is scaled up due to reduced impact of sampling and collection losses.

### Conclusion

The microbial enhancement of DDGS to increase its protein concentration and nutritional value has shown significant potential in this first phase of research. The process has been simplified to reduce cost and increase product performance.

The process has demonstrated the ability to increase the protein concentration by over 36% (31.93% to 43.43%) in large scale trials and some bench trials have shown protein levels over 50%. These results are important to the feasibility of using DDGS as an aquafeed ingredient because of the high protein requirements in aquafeeds. The process will benefit from additional optimization to ensure further increases in protein levels and performance. the critical factors for optimization have been identified in this Year-1 research and work has been begun on their implementation.

The performance of the HP-DDGS has been shown to be improved over commodity DDG and even over specialty DDGS products like StillPro or Novameal. The combination of StillPro or Novameal with the microbial conversion process offers potential for further improvement and even higher protein levels. The combination of these processes has begun and will be a part of the process optimization.

The technology to microbially enhance the protein in DDGS to develop a fish meal replacement has been demonstrated to be technically feasible, economically attractive, and a sustainable solution to increased need for quality protein ingredients to replace fishmeal in aquaculture feeds.

### Example 2

### Hybrid Solid State Fermentation (hybrid-SSF) Trials in the Omcan Reactor (˜100 L)

A feed stock was selected from the following list: soybean meal (SBM), extruded soy bean meal, DDGS, extruded white flake, or Novita Novameal. Then a 15% solid loading rate of the feedstock was added to a submerged bioreactor with distilled water to reach a total of 5 L. magrabar antifoam (2 ml) was added, and the pH was adjusted to the desired level (typically 3-5) using concentrated sulfuric acid. After autoclaving at 121° C. for 30 minutes, the material was cooled to 1) 50° C. if a saccharification phase was to be conducted or 2) 30° C. if the saccharification phase was omitted. When saccharification was used, Novozyme enzymes Htec2 (3 ml) and Ctec (5 ml) were added and the slurry was agitated at 200 rpm for 24 hr. After cooling to 30° C., the slurry was inoculated with 50 ml of a 24 culture of inoculum grown on a 5% glucose, 0.5% yeast extract medium. Cultures tested included: A. pullulans sp. 42023, A. pullulans sp. 58522, or A. pullulans sp Y-2311-1. An antibacterial gent was also added (e.g., FERMASURE or Lactrol). Incubation proceeded at 200 rpm for 24 hours before being used to inoculate the solid phase substrate.

The OMCAN (Mississauga, ON, Canada) reactor was initially disinfected and then the feedstock, water, sulfuric acid, and the antibacterial agent were added to achieve a solid loading of 50% and pH of about 3. The contents of the OMCAN were incubated at room temperature for 120 hours, with twice daily missing at 100 rpm for 30 minutes. Samples were taken every 24 hours and monitored for dry weight, pH, microbial counts, sugars, and proteins. A smaller sample was placed in a 15 ml conical tube with 5 ml of water and used for streaking plates, gram satins, pH and HPLC analysis. After incubation, the remaining contents were dried down, ground and analyzed as above.

### Results

TABLE 8
Results from Hybrid Solid State Trials.

| | | | | Protein |
|---|---|---|---|---|
| | | | | Content |
| Trial | | | | (dry matter |
| No. | Feedstock | Treatment | Organism | basis) |
| 1. | Novita | No | A. Pullulans | 43.89% |
| | DDGS | saccharification, | NRRL 58522 | |
| | | no antimicrobials, | | |
| | | incubated at pH 3, | | |
| | | 10,000 ppm nitrogen | | |
| | | supplementation*, | | |
| | | 100 rpm for 30 min | | |
| | | 2x per day | | |
| 2. | DDGS | No | A. Pullulans | 41.31% |
| | | saccharification, | NRRL 58522 | |
| | | incubation at pH 3, | | |
| | | no antimicrobial, | | |
| | | 10,000 ppm nitrogen | | |
| | | supplementation, | | |
| | | no antimicrobial | | |
| | | 100 rpm for 30 min | | |
| | | 2x per day | | |
| 3. | Ext | No | A. Pullulans | 55.32% |
| | white | saccharification, | NRRL 58522 | |
| | flake | incubation at pH 3, | | |
| | | no nitrogen | | |
| | | supplementation, | | |
| | | no antimicrobial, | | |
| | | 100 rpm for 30 min | | |
| | | 2 x per day | | |
| 4. | Extruded | No | A. Pullulans | 56.74% |
| | SBM | saccharification, | NRRL 58522 | |
| | | incubation at pH 3, | | |
| | | no nitrogen | | |
| | | supplementation, | | |
| | | no antimicrobials, | | |
| | | 100 rpm for 30 min | | |
| | | 2 x per day | | |
| 5. | SBM | No | A. Pullulans | 55.40% |
| | | saccharification, | NRRL 58522 | |
| | | incubation at pH 3, | | |
| | | no nitrogen | | |
| | | supplementation, | | |
| | | no antimicrobials, | | |
| | | 100 rpm for 30 min | | |
| | | 2 x per day | | |
| 6. | SBM | No | A. pullulans | 53.94% |
| | | saccharification, | Y-2311-1 | |
| | | incubation at pH 4, | | |
| | | no nitrogen | | |
| | | supplementation, | | |
| | | no antimicrobials, | | |
| | | continuous agitation | | |
| | | at 5 rpm | | |

*Ammonium sulfate, urea or ammonium chloride.

As can be seen in Table 8, no saccharification is required to achieve protein contents above 50% (compare for example data of Table 4) using the hybrid SSF method.

### Performance Evaluation of Hybrid-SSF HQSPC as Fish Meal Replacement in Perch Fish

Several difference among commercially available SPC were previously identified, principally in protein and amino acid composition and anti-nutritional properties, which imparted variability in growth performance and fish composition. Those experiments justified the need to develop higher quality SPC products that would support growth performance equivalent to or better than diets containing fish meal. A feeding trial will be conducted utilizing yellow perch to provide assessment of the hybrid-SSF HQSPC soy products in comparison to a commercial SPC and a Menhaden fish meal control.

Approximately 12 kg of each diet will be prepared, including 2 kg containing 1 g/100 g chromic oxide for digestability determinations. The trial diets are formulated to contain equivalent SPC amounts with an appropriate protein:lipid target of 42:10. Soy Protein Concentrate (SPC, e.g., from Solae, St. Louis, Mo. or Netzcon Ltd. Rehovot, Israel) with a minimum protein content of 69% is made by aqueous alcohol extraction of defatted non-toasted white flakes. SPC is distinctly different from soybean meal, as it contains traces of oligopolysaccharides and antigenic substances glycinin and b-conglycinin.

Large particle ingredients are ground with a Fitzpatric comminutor (Elhurst, Ill.) with 0.51 mm screen prior to dry blending. Dry ingredients are blended for 20 min using a VI-10 mixer with an intensifier bar (Vanguard Pharmaceutical Machinery, Inc., Spring, Tex.). Dry blended feedstuffs are then transferred to a Hobart HL200 mixer (Troy, Ohio) where oils and water are added and blended for about 5 min. Feeds are then screw pressed using a Hobart 4146 grinder with a 3/6″ die and dried under cool, forced-air conditions. Following drying, feeds are milled into pellets using a food processor, sieved to achieve consistent pellet size, and placed in frozen storage at −20° C.

### Pellet Properties

Samples of each diet are analyzed in triplicate for moisture (%), water activity (a_{w}), unit density (kg/m3), pellet durability index (%), water stability (min), and color (L, a, b); compressive strength (g), and diameter (mm) are determined with n=10 replications. Moisture (%) is obtained using standard method 2.2.2.5 (NFTA, 2001). Water activity (a_{w}) of 2 g pellet samples is measured with a Lab Touch a_{w} analyzer (Nocasina, Lachen SZ, Switzerland). Three color variables are analyzed with a spectrophotocolorimeter (LabScan XE, Hunter Lab, Reston, Va.) as Hunter L (brightness/darkness), Hunter a (redness greenness) and Hunter b (yellowness/blueness). Unit density (UD) is estimated by weighing 100 ml of pellets and dividing the mass (kg) by 0.0001 m³. Pellet durability index (PDI) is determined according to standard method S269.4 (ASAE 2003). The PDI is calculated as: PDI (%)=(Mₐ/M_{b})×100, where Mₐ is the mass (g) after tumbling and M_{b} is the mass (g) before tumbling. Pellet stability (min) is determined by the static (W_{static}) method (Ferouz et al., Cereal Chem (2011) 88:179-188) to mimic pellet leaching in tanks until they are consumed. Stability is calculated as loss of weight from leaching/dry weight of initial sample. Pellet diameter is measured using a conventional caliper. Pellets are tested for compressive strength using a TA.XT Plus Texture Analyzer (Scarsdale, N.Y.).

### Feeding Trial

Yellow perch (2.95 g±0.05 SE) are randomly stocked at 21 fish/tank into 28 circular tanks (110 liters) connected in parallel to a closed-loop recirculating aquaculture system (RAS). The RAS water flow and quality is maintained with a centrifugation pump consisting of dual solids sup tanks, bioreactor, bead filter, UV filter, and heat pump. System water is municipal that is dechlorinated and stored in a 15,200 L tank. Four replications of each treatment will be applied randomly in tanks. Water flow is maintained at ˜1.5 L/min/tank. Temperature is maintained at 22° C.±1°. Temperature and dissolved oxygen are measured with a YSI Pro Plus (Yellow Springs Instrument Company, Yellow Springs, Ohio). Ammonia-nitrogen, nitrite-nitrogen, nitrate-nitrogen, alkalinity (as CaCO₃), and free chlorine are tested using a Hach DR 3900 Spectrophotometer (Hach Company, Loveland, Colo.).

Fish are fed to satiation by hand twice daily, and feeding rates are modified according to tank weights, observed growth rates, and feed consumption assessments. Consumption (%) is estimated from a known number of pellets fed and by counting uneaten pellets 30 min after feeding. Collections of uneaten feed with subsequent dry weights are also used to estimate consumption. Weekly tank consumption estimates are multiplied by weekly rations to obtain weekly consumption (g). Palatability of treatments is determined by the amount of feed consumed or rejected. Tank mass (+0.01 g) is measured every other week to adjust feed rates and calculate performance indices. Individual lengths (mm) and weights (+0.01 g) are also measured every other week on four randomly sampled fish from each treatment.

Feed conversion ratio (FCR) is calculated as:

$$F C R = \frac{mass of feed consumed ( dry , g )}{growth ( wet , g )}$$

Protein conversion ratio is calculated as:

$$P E R = \frac{growth ( wet , g )}{mass of protein consumed ( dry , g )}$$

Fulton-type condition factor (K) is calculated as:

$$K = \frac{weight ( g )}{{[ length ( mm ) ]}^{3}} \times 10 , 000.$$

Specific growth rate (SGR) is calculated as:

$$S G R = \frac{[ ln ( final wt ( g ) ) - ln ( start wt ( g ) ) ] \times 100}{n ( days )}$$

Statistical analyses of diets and feeding trial responses are carried out with analysis of variance (ANOVA, a priori α=0.05). Significant F tests are followed by a post hoc Tukey's test.

### Other Assays

End of trial analyses may include final growth, FCR, PER, consumption, and examination for nutritional deficiencies via necropsy. Plasma assays may be completed for lysine and methionine using standard methods. Individual fish may be euthanized by cervical dislocation in order to quantify muscle ratio, hepatosomatic index, viscerosomatic index, fillet composition, and hind gut histology (enteritis inflammation scores). Protein and energy availability of trial diets may be estimated using chromic oxide (CrO₃) marker within the feed and fecal material (Austreng E, Aquaculture (1978) 13:265-272). Fecal material may be collected via necropsy from the lower intestinal tract.

The apparent digestibility coefficients (ADC) for the nutrients in the test diets may be calculated using the following formula:

$${ADC}_{test ingredient} = {ADC}_{test diet} + [ ( {ADC}_{test diet} - {ADC}_{ref diet} ) \times ( \frac{0.7 \times {D}_{ref}}{0.3 \times {D}_{ingr}} ) ]$$

where Dref=% with nutrient (kJ/g gross energy) of reference diet mash (as is) and Dingr=% nutrient (kJ/g gross energy) of test ingredient (as is).

### Example 3

### Production of PUFA Using Microbial Conversion

Expeller extracted soybean meal with about 5% fat remaining was used. The moisture content of the material as received was about 10%. The pH and moisture content of the soybean meal was adjusted by premixing the appropriate amount of water and acid. As an example, 8.8 kilograms of soybean meal was measured out. Separately 410 grams of concentrated sulfuric acid was mixed into 6 liters of water. The meal and acid solution were then mixed together thoroughly in a horizontal paddle mixer. The pH was then verified to be close to the target of 3.0. Then next step was to add 1 liter of prepared T. aureum inoculum and mix thoroughly again. The mixer was set on a timer so that it would mix for 5 minutes every 3 hours. The fermentation process was allowed to proceed for 144 hours. The material was dried down in a low temperature oven and saved for analysis.

All of the references cited herein are incorporated by reference in their entireties.

From the above discussion, one skilled in the art can ascertain the essential characteristics of the invention, and without departing from the spirit and scope thereof, can make various changes and modifications of the embodiments to adapt to various uses and conditions. Thus, various modifications of the embodiments, in addition to those shown and described herein, will be apparent to those skilled in the art from the foregoing description. Such modifications are also intended to fall within the scope of the appended claims.

We claim:

1. A method of producing a non-animal based protein concentrate comprising:
inoculating a substantially dry substrate selected from the group consisting of cereal grains, bran, sawdust, peat, oil-seed materials, wood chips, and combinations thereof;
subjecting the inoculated substrate to solid state fermentation (SSF) with a microbe selected from the group consisting of Aureobasidium pullulans, Fusarium venenatum, Sclerotium glucanicum, Sphingomonas paucimobilis, Ralstonia eutropha, Rhodospirillum rubrum, Issatchenkia spp, Aspergillus spp, Kluyveromyces and Pichia spp, Trichoderma reesei, Pleurotus ostreatus, Rhizopus spp, and combinations thereof;
incubating the inoculated substrate at a pH of less than 2 to about 3 or at a pH of greater than about 8; and
recovering the resulting proteins and microbes.

2. The method of claim 1, further comprising mixing the microbe and substrate to form a substantially stable pellet or billet, wherein said pellet or billet contains sufficient void volume within and between pellets or billets to allow for aeration and humidification of the stabilized substrate-microbe mixture with substantially no agitation.

3. The method of claim 1, wherein the microbe is A. pullulans.

4. The method of claim 1, wherein the substrate is non-extruded DDGS or non-extruded DDG.

5. A protein concentrate produced by the method of claim 1, wherein the protein content is between about 40 to about 50% (dry matter basis).

6. A composition comprising the protein concentrate of claim 5, which composition is a complete replacement for animal based fishmeal in a fish feed.

7. A method of producing a non-animal based protein concentrate comprising:
a) forming a feedstock and transferring feedstock to a first bioreactor;
b) inoculating the feedstock with at least one microbe in an aqueous medium, wherein said microbe converts released sugars into proteins and exopolysaccharides and optionally releases enzymes into the bulk fluid;
c) mixing the liquid in step (b) with an acid and optionally one or more antimicrobials;
d) mixing additional solids to the mixture of step (c) to reduce the moisture level of the mixture of step (c) to about 40 to about 60% and transferring said reduced moisture mixture to a second bioreactor,
wherein the mixture of step (d) is incubated in said second bioreactor for a sufficient time to convert the solids into said protein concentrate.

8. The method of claim 7, wherein step (b) is carried out at about 30 to about 50° C. for about 24 hours.

9. The method of claim 7, wherein step (d) is carried out at about 25° C. for about 5 days.

10. The method of claim 7, wherein the microbe is a fungus.

11. The method of claim 10, wherein the fungi is Aureobasidium pullulans.

12. The method of claim 8, further comprising supplementing the inoculum with a nitrogen source.

13. The method of claim 12, wherein said nitrogen source is selected from the group consisting of ammonium sulfate, urea, and ammonium chloride.

14. The method of claim 7, wherein the second bioreactor is conical or tubular.

15. The method of claim 7, wherein the fermentation is carried out in the absence of exogenous saccharifying enzymes.

16. A protein concentrate produced by the method of claim 7, wherein the protein content is between about 50 to about 60% (dry matter basis).

17. A composition comprising the protein concentrate of claim 16, which composition is a complete replacement for animal based fishmeal in a fish feed.

18. A method of producing polyunsaturated fatty acid (PUFA) comprising:
inoculating a substrate containing low PUFA lipids either as provided or by addition, wherein the substrate is selected from the group consisting of cereal grains, bran, sawdust, peat, oil-seed materials, wood chips, syrup, and combinations thereof;
subjecting the inoculated substrate to solid state fermentation (SSF) with a microbe selected from the group consisting of Pythium, Thraustochytrium and Schizochytrium, and combinations thereof;
incubating the inoculated substrate.

19. The method of claim 18, further comprising adding the resulting PUFA enhanced material as an ingredient in an animal feed or alternatively recovering the resulting PUFA enhanced lipids.

20. A composition comprising the produce of the method of claim 18, wherein the lipid of the composition has about 50-95% triacylglycerol content.
